SEQUENCE LISTING (70 sequences)

SEQ ID NO: 1 - protein, 452 aa, synthetic construct
EVQLVESGGGVVQPGRSLRLSCAASGFTFTDYYMSWVRQAPGKGLEWVAMSRNKAKGHTIEYSSSVKGRFTISRDNSKNTLYLQMNSLRAEDTAVYYCARDTYESYDGYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 219 aa, synthetic construct
DIVMTQSPDSLAVSLGERATINCKSSQSLFNSRSRKNYLAWYQQKPGQTPKLLIYWASIRESGVPDRFSGSGSGTDFTLTISSLQAEDVAVYYCKQSYYLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 353 aa, synthetic construct
EVQLVESGGGLVQPGRSLRLSCAASGDITRRCMGWFRQAPGKEREGVAGISEDGITIYADSVKGRFTISKDNAKNSLYLQMNSLRAEDTALYSCAASILNRVGCRQLSYEYIYRGQGTMVTVSSGGGDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 4 - protein, 352 aa, synthetic construct
EVQLVESGGGVVQPGGSLRLSCAASGDTFSRGSWGWFRQAPGKEREAVALISSDGWTFYADSVKGRFTISQDNSKNTLYLQMNSLRAEDTAVYYCAASVLVLRTLNPKAWHHWGQGTMVTVSSGGGDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 5 - protein, 352 aa, synthetic construct
EVQLVESGGGLVQPGGSLRLSCAASGFPSSRGTMAWFRQAPGKEREGVAFINSDGRTSYAESVKGRFTISRDNAKNSLYLQMNSLRAEDTAVYYCAASLLYLMTVNAAAYPYWGQGTMVTVSSGGGDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 6 - protein, 353 aa, synthetic construct
EVQLVESGGGVVQPGGSLRLSCAASGYTSNRYSMAWFRQAPGKEREVVAFIWTDGGRTYYADSVKGRFTISRDNSKNTLYLQMNSLRAEDTAVYYCAASSLLLQTINPRAWPYWGQGTMVTVSSGGGDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 7 - protein, 474 aa, synthetic construct
EVKLDETGGGLVQPGRPMKLSCVASGFTFSDYWMNWVRQSPEKGLEWVAQIRNKPYNYETYYSDSVKGRFTISRDDSKSSVYLQMNNLRVEDMGIYYCTGSYYGMDYWGQGTSVTVSSGGGGSGGGGSGGGGSDVVMTQTPLSLPVSLGDQASISCRSSQSLVHSNGNTYLRWYLQKPGQSPKVLIYKVSNRFSGVPDRFSGSGSGTDFTLKISRVEAEDLGVYFCSQSTHVPWTFGGGTKLEIKGGGDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 8 - protein, 984 aa, synthetic construct
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSSGGGGSGGGGSGGGGSSYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 123 aa, synthetic construct
EVQLVESGGGVVQPGRSLRLSCAASGFTFTDYYMSWVRQAPGKGLEWVAMSRNKAKGHTIEYSSSVKGRFTISRDNSKNTLYLQMNSLRAEDTAVYYCARDTYESYDGYFDVWGQGTTVTVSS

SEQ ID NO: 10 - protein, 112 aa, synthetic construct
DIVMTQSPDSLAVSLGERATINCKSSQSLFNSRSRKNYLAWYQQKPGQTPKLLIYWASIRESGVPDRFSGSGSGTDFTLTISSLQAEDVAVYYCKQSYYLYTFGQGTKLEIK

SEQ ID NO: 11 - protein, 124 aa, synthetic construct
EVQLVESGGGLVQPGRSLRLSCAASGDITRRCMGWFRQAPGKEREGVAGISEDGITIYADSVKGRFTISKDNAKNSLYLQMNSLRAEDTALYSCAASILNRVGCRQLSYEYIYRGQGTMVTVSS

SEQ ID NO: 12 - protein, 123 aa, synthetic construct
EVQLVESGGGVVQPGGSLRLSCAASGDTFSRGSWGWFRQAPGKEREAVALISSDGWTFYADSVKGRFTISQDNSKNTLYLQMNSLRAEDTAVYYCAASVLVLRTLNPKAWHHWGQGTMVTVSS

SEQ ID NO: 13 - protein, 123 aa, synthetic construct
EVQLVESGGGLVQPGGSLRLSCAASGFPSSRGTMAWFRQAPGKEREGVAFINSDGRTSYAESVKGRFTISRDNAKNSLYLQMNSLRAEDTAVYYCAASLLYLMTVNAAAYPYWGQGTMVTVSS

SEQ ID NO: 14 - protein, 124 aa, synthetic construct
EVQLVESGGGVVQPGGSLRLSCAASGYTSNRYSMAWFRQAPGKEREVVAFIWTDGGRTYYADSVKGRFTISRDNSKNTLYLQMNSLRAEDTAVYYCAASSLLLQTINPRAWPYWGQGTMVTVSS

SEQ ID NO: 15 - protein, 5 aa, synthetic construct
DYYMS

SEQ ID NO: 16 - protein, 19 aa, synthetic construct
MSRNKAKGHTIEYSSSVKG

SEQ ID NO: 17 - protein, 12 aa, synthetic construct
DTYESYDGYFDV

SEQ ID NO: 18 - protein, 17 aa, synthetic construct
KSSQSLFNSRSRKNYLA

SEQ ID NO: 19 - protein, 7 aa, synthetic construct
WASIRES

SEQ ID NO: 20 - protein, 8 aa, synthetic construct
KQSYYLYT

SEQ ID NO: 21 - protein, 4 aa, synthetic construct
RCMG

SEQ ID NO: 22 - protein, 16 aa, synthetic construct
GISEDGITIYADSVKG

SEQ ID NO: 23 - protein, 17 aa, synthetic construct
SILNRVGCRQLSYEYIY

SEQ ID NO: 24 - protein, 5 aa, synthetic construct
RGSWG

SEQ ID NO: 25 - protein, 16 aa, synthetic construct
LISSDGWTFYADSVKG

SEQ ID NO: 26 - protein, 15 aa, synthetic construct
SVLVLRTLNPKAWHH

SEQ ID NO: 27 - protein, 5 aa, synthetic construct
RGTMA

SEQ ID NO: 28 - protein, 16 aa, synthetic construct
FINSDGRTSYAESVKG

SEQ ID NO: 29 - protein, 15 aa, synthetic construct
SLLYLMTVNAAAYPY

SEQ ID NO: 30 - protein, 5 aa, synthetic construct
RYSMA

SEQ ID NO: 31 - protein, 17 aa, synthetic construct
FIWTDGGRTYYADSVKG

SEQ ID NO: 32 - protein, 15 aa, synthetic construct
SSLLLQTINPRAWPY

SEQ ID NO: 33 - protein, 7 aa, synthetic construct
GFTFTDY

SEQ ID NO: 34 - protein, 8 aa, synthetic construct
RNKAKGHT

SEQ ID NO: 35 - protein, 12 aa, synthetic construct
DTYESYDGYFDV

SEQ ID NO: 36 - protein, 17 aa, synthetic construct
KSSQSLFNSRSRKNYLA

SEQ ID NO: 37 - protein, 7 aa, synthetic construct
WASIRES

SEQ ID NO: 38 - protein, 8 aa, synthetic construct
KQSYYLYT

SEQ ID NO: 39 - protein, 6 aa, synthetic construct
GDITRR

SEQ ID NO: 40 - protein, 5 aa, synthetic construct
SEDGI

SEQ ID NO: 41 - protein, 17 aa, synthetic construct
SILNRVGCRQLSYEYIY

SEQ ID NO: 42 - protein, 7 aa, synthetic construct
GDTFSRG

SEQ ID NO: 43 - protein, 5 aa, synthetic construct
SSDGW

SEQ ID NO: 44 - protein, 15 aa, synthetic construct
SVLVLRTLNPKAWHH

SEQ ID NO: 45 - protein, 7 aa, synthetic construct
GFPSSRG

SEQ ID NO: 46 - protein, 5 aa, synthetic construct
NSDGR

SEQ ID NO: 47 - protein, 15 aa, synthetic construct
SLLYLMTVNAAAYPY

SEQ ID NO: 48 - protein, 7 aa, synthetic construct
GYTSNRY

SEQ ID NO: 49 - protein, 6 aa, synthetic construct
WTDGGR

SEQ ID NO: 50 - protein, 15 aa, synthetic construct
SSLLLQTINPRAWPY

SEQ ID NO: 51 - protein, 8 aa, synthetic construct
GFTFTDYY

SEQ ID NO: 52 - protein, 10 aa, synthetic construct
SRNKAKGHTI

SEQ ID NO: 53 - protein, 14 aa, synthetic construct
ARDTYESYDGYFDV

SEQ ID NO: 54 - protein, 12 aa, synthetic construct
QSLFNSRSRKNY

SEQ ID NO: 55 - skipped

SEQ ID NO: 56 - protein, 8 aa, synthetic construct
KQSYYLYT

SEQ ID NO: 57 - protein, 7 aa, synthetic construct
GDITRRC

SEQ ID NO: 58 - protein, 7 aa, synthetic construct
ISEDGIT

SEQ ID NO: 59 - protein, 19 aa, synthetic construct
AASILNRVGCRQLSYEYIY

SEQ ID NO: 60 - protein, 8 aa, synthetic construct
GDTFSRGS

SEQ ID NO: 61 - protein, 7 aa, synthetic construct
ISSDGWT

SEQ ID NO: 62 - protein, 17 aa, synthetic construct
AASVLVLRTLNPKAWHH

SEQ ID NO: 63 - protein, 8 aa, synthetic construct
GFPSSRGT

SEQ ID NO: 64 - protein, 7 aa, synthetic construct
INSDGRT

SEQ ID NO: 65 - protein, 17 aa, synthetic construct
AASLLYLMTVNAAAYPY

SEQ ID NO: 66 - protein, 8 aa, synthetic construct
GYTSNRYS

SEQ ID NO: 67 - protein, 8 aa, synthetic construct
IWTDGGRT

SEQ ID NO: 68 - protein, 17 aa, synthetic construct
AASSLLLQTINPRAWPY

SEQ ID NO: 69 - protein, 269 aa, synthetic construct
RTTTCFGDGCQNTASRCKNGGTWDGLKCQCPNLYYGELCEEVVSSIDIGPPETISAQMELTVTVTSVKFTEELKNHSSQEFQEFKQTFTEQMNIVYSGIPEYVGVNITKLRLGSVVVEHDVLLRTKYTPEYKTVLDNATEVVKEKITKVTTQQIMINDICSDMMCFNTTGTQVQNITVTQYDPEEDCRKMAKEYGDYFVVEYRDQKPYCISPCEPGFSVSKNCNLGKCQMSLSGPQCLCVTTETHWYSGETCNQGTQKSLVYGHHHHHH

SEQ ID NO: 70 - protein, 518 aa, synthetic construct
PLATIPVSTTSLTSSEGSTISTPSVDISTPVTTSIVGDQCSRCKNGGFWDGLKCQCLTPYYGESCEEVVNSIDIAPPETVSAQMELTVTVTSVKFTDELKNHSSQEFREFNKTFTEQMNIVYSGIPEYVGVNITNLRLGSVVVEHDVLLRTKYTPEYKTALDNATEVVKQKITKVTTEQIMTNDNCSALMCFNTTGTQVQNITVTQYDPVEECRQKAEEYEDYFLVEYRDQKPYCISPCESGFNASKNCNHGKCQMSQNGARCLCVTTETHWYSGEDCNQGTQKSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

(19) United States
(12) Patent Application Publication
(10) Pub. No.: US 2026/0226191 A1
(43) Pub. Date: Aug. 6, 2026

(54) BISPECIFIC ANTIBODY AGAINST MUC17 AND CD3, AND USE THEREOF

(71) Applicant: SHANDONG SIMCERE BIOPHARMACEUTICAL CO., LTD., Yantai, Shandong (CN)

(72) Inventors: Guangcun CHENG, Shanghai (CN); Yayuan FU, Shanghai (CN); Renhong TANG, Shanghai (CN)

(21) Appl. No.: 19/153,237

(22) PCT Filed: Feb. 2, 2024
(86) PCT No.: PCT/CN2024/075414

§ 371 Date: Aug. 1, 2025

(30) Foreign Application Priority Data
Feb. 3, 2023 (CN) 202310092717.2
Mar. 23, 2023 (CN) 202310308001.1

(51) Int. Cl. C07K 16/30; A61K 39/00; A61K 45/06; A61P 35/00; C07K 16/28; C12N 15/63

(52) U.S. Cl. CPC C07K 16/3092; A61K 45/06; A61P 35/00; C07K 16/2809; C12N 15/63; A61K 2039/505; C07K 2317/14; C07K 2317/24; C07K 2317/31; C07K 2317/565; C07K 2317/569; C07K 2317/92

(57) ABSTRACT

The present invention relates to a bispecific antibody capable of specifically binding to MUC17 and CD3, wherein the bispecific antibody can modulate an immune cell function, and can be used as a drug for treatment of gastrointestinal malignant tumor. Additionally, further provided are a polynucleotide encoding the bispecific antibody, a vector, a host cell, and a pharmaceutical composition comprising the bispecific antibody.

### CROSS-REFERENCE TO RELATED APPLICATIONS

The present application claims priority and benefit to the Chinese Application No. 202310092717.2 filed on Feb. 3, 2023 and the Chinese Application No. 202310308001.1 filed on Mar. 23, 2023, which are incorporated herein in their entirety for all purposes.

### TECHNICAL FIELD

The present application relates to the field of antibodies, in particular to a bispecific antibody against MUC17 and CD3.

### BACKGROUND

Bispecific antibodies (BsAbs) are a class of antibody molecules that can simultaneously bind to two different antigens or two different epitopes on the same antigen. The two unique antigen-binding sites are capable of advantageously achieving the binding to two targets, thereby exerting the synergistic effect of the two monoclonal antibodies. The bispecific antibodies can act as a bridge between target cells and functional molecules, thereby producing a guided effector function. The bispecific antibodies may have more advantages than the combination of two monoclonal antibodies in preclinical research and clinical treatment, and have broad application prospects in the fields of tumor immunotherapy, autoimmune diseases, and the like.

The killing of tumor cells through the cytotoxicity mediated by bispecific antibodies is a hot spot of current immunotherapeutic application research, which is mainly achieved by the fact that the bispecific antibodies are capable of simultaneously binding to effector cells and tumor-associated antigens, thereby directly triggering the specific killing of tumor cells by immune cells. CD3 is a surface-specific molecule present on all T lymphocytes, through which effector T cells with killing effects can be recruited. MUC17 is a tumor-specific antigen and is widely expressed in a variety of tumor cells, but is only expressed in intestinal tissues among normal tissues. Therefore, the specific killing of MUC17-positive tumors can be achieved by targeting CD3 and MUC17.

### SUMMARY

The present application provides an anti-MUC17/anti-CD3 bispecific antibody, a nucleic acid molecule for encoding the antibody, an antibody preparation method, a pharmaceutical composition comprising the antibody, and related use of the pharmaceutical composition in treating a tumor.

In a first aspect, the present application provides an anti-MUC17/anti-CD3 bispecific antibody, which comprises:
(a) a first antigen-binding moiety comprising a heavy chain variable region (VH) and a light chain variable region (VL), wherein the VH and VL form an anti-CD3 antigen-binding domain, wherein the anti-CD3 antigen-binding domain comprises an HCDR1, an HCDR2, and an HCDR3 in a VH set forth in SEQ ID NO. 9, and an LCDR1, an LCDR2, and an LCDR3 in a VL set forth in SEQ ID NO. 10;
(b) a second antigen-binding moiety comprising a VHH specifically binding to MUC17, wherein the VHH comprises a CDR1, a CDR2, and a CDR3 from the sequence set forth in SEQ ID NO. 11, or a CDR1, a CDR2, and a CDR3 from the sequence set forth in SEQ ID NO. 12, or a CDR1, a CDR2, and a CDR3 from the sequence set forth in SEQ ID NO. 13, or a CDR1, a CDR2, and a CDR3 from the sequence set forth in SEQ ID NO. 14.

In some embodiments, based on the Kabat numbering scheme, in the first antigen-binding moiety, the HCDR1 comprises the sequence set forth in SEQ ID NO. 15; the HCDR2 comprises the sequence set forth in SEQ ID NO. 16; and the HCDR3 comprises the sequence set forth in SEQ ID NO. 17.

In some embodiments, based on the Chothia numbering scheme, in the first antigen-binding moiety, the HCDR1 comprises the sequence set forth in SEQ ID NO. 33; the HCDR2 comprises the sequence set forth in SEQ ID NO. 34; and the HCDR3 comprises the sequence set forth in SEQ ID NO. 35.

In some embodiments, based on the IMGT numbering scheme, in the first antigen-binding moiety, the HCDR1 comprises the sequence set forth in SEQ ID NO. 51; the HCDR2 comprises the sequence set forth in SEQ ID NO. 52; and the HCDR3 comprises the sequence set forth in SEQ ID NO. 53.

In some embodiments, based on the Kabat numbering scheme, in the first antigen-binding moiety, the LCDR1 comprises the sequence set forth in SEQ ID NO. 18; the LCDR2 comprises the sequence set forth in SEQ ID NO. 19; and the LCDR3 comprises the sequence set forth in SEQ ID NO. 20.

In some embodiments, based on the Chothia numbering scheme, in the first antigen-binding moiety, the LCDR1 comprises the sequence set forth in SEQ ID NO. 36; the LCDR2 comprises the sequence set forth in SEQ ID NO. 37; and the LCDR3 comprises the sequence set forth in SEQ ID NO. 38.

In some embodiments, based on the IMGT numbering scheme, in the first antigen-binding moiety, the LCDR1 comprises the sequence set forth in SEQ ID NO. 54; the LCDR2 comprises the sequence set forth in SEQ ID NO. 55; and the LCDR3 comprises the sequence set forth in SEQ ID NO. 56.

In some embodiments, in the first antigen-binding moiety, the HCDR1 comprises the sequence set forth in any one of SEQ ID NOs. 15, 33, and 51; the HCDR2 comprises the sequence set forth in any one of SEQ ID NOs. 16, 34, and 52; and the HCDR3 comprises the sequence set forth in any one of SEQ ID NOs. 17, 35, and 53.

In some embodiments, in the first antigen-binding moiety, the LCDR1 comprises the sequence set forth in any one of SEQ ID NOs. 18, 36, and 54; the LCDR2 comprises the sequence set forth in any one of SEQ ID NOs. 19, 37, and 55; and the LCDR3 comprises the sequence set forth in any one of SEQ ID NOs. 20, 38, and 56.

In some embodiments, based on the Kabat numbering scheme, in the second antigen-binding moiety, the CDR1 comprises the sequence set forth in SEQ ID NO. 21; the CDR2 comprises the sequence set forth in SEQ ID NO. 22; and the CDR3 comprises the sequence set forth in SEQ ID NO. 23.

In some embodiments, based on the Kabat numbering scheme, in the second antigen-binding moiety, the CDR1 comprises the sequence set forth in SEQ ID NO. 24; the CDR2 comprises the sequence set forth in SEQ ID NO. 25; and the CDR3 comprises the sequence set forth in SEQ ID NO. 26.

In some embodiments, based on the Kabat numbering scheme, in the second antigen-binding moiety, the CDR1 comprises the sequence set forth in SEQ ID NO. 27; the CDR2 comprises the sequence set forth in SEQ ID NO. 28; and the CDR3 comprises the sequence set forth in SEQ ID NO. 29.

In some embodiments, based on the Kabat numbering scheme, in the second antigen-binding moiety, the CDR1 comprises the sequence set forth in SEQ ID NO. 30; the CDR2 comprises the sequence set forth in SEQ ID NO. 31; and the CDR3 comprises the sequence set forth in SEQ ID NO. 32.

In some embodiments, based on the Chothia numbering scheme, in the second antigen-binding moiety, the CDR1 comprises the sequence set forth in SEQ ID NO. 39; the CDR2 comprises the sequence set forth in SEQ ID NO. 40; and the CDR3 comprises the sequence set forth in SEQ ID NO. 41.

In some embodiments, based on the Chothia numbering scheme, in the second antigen-binding moiety, the CDR1 comprises the sequence set forth in SEQ ID NO. 42; the CDR2 comprises the sequence set forth in SEQ ID NO. 43; and the CDR3 comprises the sequence set forth in SEQ ID NO. 44.

In some embodiments, based on the Chothia numbering scheme, in the second antigen-binding moiety, the CDR1 comprises the sequence set forth in SEQ ID NO. 45; the CDR2 comprises the sequence set forth in SEQ ID NO. 46; and the CDR3 comprises the sequence set forth in SEQ ID NO. 47.

In some embodiments, based on the Chothia numbering scheme, in the second antigen-binding moiety, the CDR1 comprises the sequence set forth in SEQ ID NO. 48; the CDR2 comprises the sequence set forth in SEQ ID NO. 49; and the CDR3 comprises the sequence set forth in SEQ ID NO. 50.

In some embodiments, based on the IMGT numbering scheme, in the second antigen-binding moiety, the CDR1 comprises the sequence set forth in SEQ ID NO. 57; the CDR2 comprises the sequence set forth in SEQ ID NO. 58; and the CDR3 comprises the sequence set forth in SEQ ID NO. 59.

In some embodiments, based on the IMGT numbering scheme, in the second antigen-binding moiety, the CDR1 comprises the sequence set forth in SEQ ID NO. 60; the CDR2 comprises the sequence set forth in SEQ ID NO. 61; and the CDR3 comprises the sequence set forth in SEQ ID NO. 62.

In some embodiments, based on the IMGT numbering scheme, in the second antigen-binding moiety, the CDR1 comprises the sequence set forth in SEQ ID NO. 63; the CDR2 comprises the sequence set forth in SEQ ID NO. 64; and the CDR3 comprises the sequence set forth in SEQ ID NO. 65.

In some embodiments, based on the IMGT numbering scheme, in the second antigen-binding moiety, the CDR1 comprises the sequence set forth in SEQ ID NO. 66; the CDR2 comprises the sequence set forth in SEQ ID NO. 67; and the CDR3 comprises the sequence set forth in SEQ ID NO. 68.

In some embodiments, in the second antigen-binding moiety, the CDR1 comprises the sequence set forth in any one of SEQ ID NOs. 21, 24, 27, 30, 39, 42, 45, 48, 57, 60, 63, and 66; the CDR2 comprises the sequence set forth in any one of SEQ ID NOs. 22, 25, 28, 31, 40, 43, 46, 49, 58, 61, 64, and 67; and the CDR3 comprises the sequence set forth in any one of SEQ ID NOs. 23, 26, 29, 32, 41, 44, 47, 50, 59, 62, 65, and 68.

In some embodiments, the first antigen-binding moiety comprises an HCDR1, an HCDR2, an HCDR3, an LCDR1, an LCDR2, and an LCDR3 of the following sequences:
(1) based on the Kabat numbering scheme, the sequences set forth in SEQ ID NOs. 15, 16, 17, 18, 19, and 20, respectively; or
(2) based on the Chothia numbering scheme, the sequences set forth in SEQ ID NOs. 33, 34, 35, 36, 37, and 38, respectively; or
(3) based on the IMGT numbering scheme, the sequences set forth in SEQ ID NOs. 51, 52, 53, 54, 55, and 56, respectively; or
(4) sequences having at least 90% identity or having 1, 2, 3, or more amino acid insertions, deletions, and/or substitutions compared with the sequences set forth in (1) to (3) described above, wherein preferably, the substitutions are conservative amino acid substitutions.

In some embodiments, the second antigen-binding moiety comprises a CDR1, a CDR2, and a CDR3 of the following sequences:
(1) based on the Kabat numbering scheme, the sequences set forth in SEQ ID NOs. 21, 22, and 23, respectively; or
(2) based on the Kabat numbering scheme, the sequences set forth in SEQ ID NOs. 24, 25, and 26, respectively; or
(3) based on the Kabat numbering scheme, the sequences set forth in SEQ ID NOs. 27, 28, and 29, respectively; or
(4) based on the Kabat numbering scheme, the sequences set forth in SEQ ID NOs. 30, 31, and 32, respectively; or
(5) based on the Chothia numbering scheme, the sequences set forth in SEQ ID NOs. 39, 40, and 41, respectively; or
(6) based on the Chothia numbering scheme, the sequences set forth in SEQ ID Nos. 42, 43, and 44, respectively; or
(7) based on the Chothia numbering scheme, the sequences set forth in SEQ ID NOs. 45, 46, and 47, respectively; or
(8) based on the Chothia numbering scheme, the sequences set forth in SEQ ID NOs. 48, 49, and 50, respectively; or
(9) based on the IMGT numbering scheme, the sequences set forth in SEQ ID NOs. 57, 58, and 59, respectively; or
(10) based on the IMGT numbering scheme, the sequences set forth in SEQ ID NOs. 60, 61, and 62, respectively; or
(11) based on the IMGT numbering scheme, the sequences set forth in SEQ ID NOs. 63, 64, and 65, respectively; or
(12) based on the IMGT numbering scheme, the sequences set forth in SEQ ID NOs. 66, 67, and 68, respectively; or
(13) sequences having at least 90% identity or having 1, 2, 3, or more amino acid insertions, deletions, and/or substitutions compared with the sequences set forth in (1) to (12) described above, wherein preferably, the substitutions are conservative amino acid substitutions.

In some embodiments, the VH of the first antigen-binding moiety comprises a sequence having at least 90% identity to the amino acid sequence set forth in SEQ ID NO. 9; the VL of the first antigen-binding moiety comprises a sequence having at least 90% identity to the amino acid sequence set forth in SEQ ID NO. 10.

In some embodiments, the second antigen-binding moiety comprises a sequence having at least 90% identity to the amino acid sequence set forth in any one of SEQ ID NOs. 11-14.

In some embodiments, the bispecific antibody comprises: a heavy chain comprising the anti-CD3 VH, a light chain comprising the anti-CD3 VL, and a heavy chain comprising the anti-MUC17 VHH.

In some embodiments, the heavy chain comprising the anti-CD3 VH comprises a sequence having at least 80% identity to the amino acid sequence set forth in SEQ ID NO. 1, the light chain comprising the anti-CD3 VL comprises a sequence having at least 80% identity to the amino acid sequence set forth in SEQ ID NO. 2, and the heavy chain comprising the anti-MUC17 VHH comprises a sequence having at least 80% identity to the amino acid sequence set forth in SEQ ID NO. 3, 4, 5, or 6.

In some embodiments, the bispecific antibody is a humanized antibody.

In some embodiments, the bispecific antibody specifically binds to human and monkey MUC17 proteins; preferably, the bispecific antibody binds to human and monkey MUC17 with a KD superior to 1.00E-9 M.

In a second aspect, the present application provides an isolated nucleic acid molecule encoding the bispecific antibody according to the first aspect.

In a third aspect, the present application provides a vector comprising the nucleic acid molecule according to the second aspect.

In a fourth aspect, the present application provides a host cell comprising the vector according to the third aspect, wherein preferably, the cell is a prokaryotic cell or a eukaryotic cell, such as a bacterium (e.g., E. coli), a fungus (e.g., yeast), an insect cell, or a mammalian cell (e.g., a CHO cell line or a 293T cell line).

In a fifth aspect, the present application provides a method for preparing the bispecific antibody according to the first aspect, which comprises culturing the host cell according to the fourth aspect, and isolating the bispecific antibody expressed by the cell.

In a sixth aspect, the present application provides a pharmaceutical composition comprising the bispecific antibody according to the first aspect, the nucleic acid molecule according to the second aspect, the vector according to the third aspect, the host cell according to the fourth aspect, or a product prepared by the method according to the fifth aspect, and a pharmaceutically acceptable carrier.

In some embodiments, the pharmaceutical composition further comprises an additional therapeutic agent; preferably, the additional therapeutic agent is an antineoplastic agent; more preferably, the antineoplastic agent is a PD-1 axis binding antagonist, a small molecule antineoplastic agent, or a cell therapeutic agent.

In a seventh aspect, the present application provides use of the bispecific antibody according to the first aspect, the nucleic acid molecule according to the second aspect, the vector according to the third aspect, the host cell according to the fourth aspect, a product prepared by the method according to the fifth aspect, or the pharmaceutical composition according to the sixth aspect in the manufacture of a medicament for treating a cancer, a tumor, or an infectious disease, wherein the cancer or tumor is selected from a solid tumor and a hematological tumor.

In some embodiments, the cancer or tumor is a MUC17-positive cancer or MUC17-positive tumor, or an MUC17 protein is expressed on the cell surface of the cancer or tumor.

In some embodiments, the cancer or tumor is selected from gastric cancer, pancreatic cancer, small intestine cancer, large intestine cancer, rectal cancer, colon cancer, colorectal cancer, esophageal cancer, breast cancer, non-small cell lung cancer, adenocarcinoma, non-Hodgkin's lymphoma (NHL), B-cell lymphoma, B-cell leukemia, multiple myeloma, renal cancer, prostate cancer, liver cancer, head and neck cancer, melanoma, ovarian cancer, mesothelioma, glioblastoma, thyroid cancer, bladder cancer, cervical cancer, blood cancer, skin cancer, epithelial cancer, brain cancer, and central nervous system cancer.

In some embodiments, the cancer is gastric cancer or pancreatic cancer.

In some embodiments, the medicament is used in combination with an additional therapeutic agent or surgery, wherein the additional therapeutic agent or surgery is selected from radiation therapy, chemotherapy, an oncolytic drug, a cytotoxic agent, a cytokine, a surgical treatment, an immunostimulatory antibody, an immunomodulatory drug, an activator of a costimulatory molecule, an inhibitor of an inhibitory molecule, a vaccine, and cellular immunotherapy.

In an eighth aspect, the present application provides a method for treating a cancer, a tumor, or an infectious disease, which comprises administering to a patient in need thereof an effective amount of the bispecific antibody according to the first aspect, the nucleic acid molecule according to the second aspect, the vector according to the third aspect, the host cell according to the fourth aspect, a product prepared by the method according to the fifth aspect, or the pharmaceutical composition according to the sixth aspect, wherein the cancer or tumor is selected from a solid tumor and a hematological tumor.

In some embodiments, the cancer or tumor is a MUC17-positive cancer or MUC17-positive tumor, or an MUC17 protein is expressed on the cell surface of the cancer or tumor.

In some embodiments, the cancer or tumor is selected from gastric cancer, pancreatic cancer, small intestine cancer, large intestine cancer, rectal cancer, colon cancer, colorectal cancer, esophageal cancer, breast cancer, non-small cell lung cancer, adenocarcinoma, non-Hodgkin's lymphoma (NHL), B-cell lymphoma, B-cell leukemia, multiple myeloma, renal cancer, prostate cancer, liver cancer, head and neck cancer, melanoma, ovarian cancer, mesothelioma, glioblastoma, thyroid cancer, bladder cancer, cervical cancer, blood cancer, skin cancer, epithelial cancer, brain cancer, and central nervous system cancer.

In some embodiments, the cancer is gastric cancer or pancreatic cancer.

In a ninth aspect, the present application provides the bispecific antibody according to the first aspect, the nucleic acid molecule according to the second aspect, the vector according to the third aspect, the host cell according to the fourth aspect, a product prepared by the method according to the fifth aspect, or the pharmaceutical composition according to the sixth aspect for use in the pretreatment of a cancer, a tumor, or an infectious disease, wherein the cancer or tumor is selected from a solid tumor and a hematological tumor.

In some embodiments, the cancer or tumor is a MUC17-positive cancer or MUC17-positive tumor, or an MUC17 protein is expressed on the cell surface of the cancer or tumor.

In some embodiments, the cancer or tumor is selected from gastric cancer, pancreatic cancer, small intestine cancer, large intestine cancer, rectal cancer, colon cancer, colorectal cancer, esophageal cancer, breast cancer, non-small cell lung cancer, adenocarcinoma, non-Hodgkin's lymphoma (NHL), B-cell lymphoma, B-cell leukemia, multiple myeloma, renal cancer, prostate cancer, liver cancer, head and neck cancer, melanoma, ovarian cancer, mesothelioma, glioblastoma, thyroid cancer, bladder cancer, cervical cancer, blood cancer, skin cancer, epithelial cancer, brain cancer, and central nervous system cancer.

In some embodiments, the cancer is gastric cancer or pancreatic cancer.

### BRIEF DESCRIPTION OF THE DRAWINGS

Unless otherwise defined herein, scientific and technical terms used in correlation with the present application shall have the meanings that are commonly understood by those skilled in the art.

FIG. 1 shows the structure construction of a bispecific antibody.

FIG. 2A shows the binding activity of Bis57, Bis58, Bis59, and Bis92 to the human MUC17 protein as detected by ELISA.

FIG. 2B shows the binding activity of Bis57, Bis58, Bis59, and Bis92 to the monkey MUC17 protein (mFc-tagged cynomolgus monkey MUC17 protein extracellular region fusion protein) as detected by ELISA.

FIG. 3A shows the binding activity of Bis57, Bis58, Bis59, and Bis92 to human CD3e-His as detected by ELISA.

FIG. 3B shows the binding activity of Bis57, Bis58, Bis59, and Bis92 to monkey CD3e-His as detected by ELISA.

FIGS. 4A to 4C show the binding reactions of Bis57, Bis58, Bis59, and Bis92 with human endogenous tumor cell lines NUGC4, SNU16, and ASPC1 as detected by FACS.

FIG. 5A shows the binding reactions of Bis57, Bis58, Bis59, and Bis92 with cells overexpressing human MUC17, FlpinCHO-huMUC17-D1, as detected by FACS.

FIG. 5B shows the binding reactions of Bis57, Bis58, Bis59, and Bis92 with cells overexpressing monkey MUC17, FlpinCHO-cynoMUC17-D1, as detected by FACS.

FIG. 6 shows the binding reactions of Bis57, Bis58, Bis59, and Bis92 with human Jurkat cells as detected by FACS.

FIGS. 7A to 7B show the binding activity of Bis57, Bis58, Bis59, and Bis92 to human and monkey PBMCs as detected by FACS.

FIG. 8A shows the activation of Bis57, Bis58, Bis59, and Bis92 after co-incubation of NUGC4 and Jurkat-luc as detected by a luciferase reporter gene assay.

FIG. 8B shows the activation of Bis57, Bis58, Bis59, and Bis92 after co-incubation of MDA-231 and Jurkat-luc as detected by a luciferase reporter gene assay.

FIG. 9 shows the activation of T cells by bispecific antibodies Bis57 and Bis59.

FIG. 10 shows the expression levels of MUC17 on the surfaces of tumor cell lines ASPC-1, NUGC4, and SNU-16 as detected by FACS.

FIGS. 11A to 11C show the evaluation of the killing activity of Bis57 and Bis59 against tumor cell lines SNU16, ASPC1, and NUGC4.

FIGS. 11D to 11E show the evaluation of the killing activity of Bis58 and Bis92 against tumor cell lines NUGC4 and ASPC1.

FIG. 12A shows the stimulation of IFNγ secretion from PBMCs by bispecific antibodies Bis57 and Bis59 in the presence of SNU16 cells.

FIG. 12B shows the stimulation of IFNγ secretion from PBMCs by bispecific antibodies Bis58 and Bis92 in the presence of ASPC1 cells.

FIG. 12C shows the stimulation of TNFα secretion from PBMCs by bispecific antibodies Bis57 and Bis59 in the presence of ASPC1 cells.

FIG. 12D shows the stimulation of TNFα secretion from PBMCs by bispecific antibodies Bis58 and Bis92 in the presence of NUGC4 cells.

FIG. 12E shows the stimulation of IL6 secretion from PBMCs by bispecific antibodies Bis57 and Bis59 in the presence of NUGC4 cells.

FIG. 12F shows the stimulation of IL6 secretion from PBMCs by bispecific antibodies Bis58 and Bis92 in the presence of ASPC1 cells.

FIG. 13 shows the pharmacokinetics in wild-type C57 mice.

FIG. 14A shows the inhibitory ability of bispecific antibodies against tumors evaluated in a PBMC reconstitution model.

FIG. 14B shows changes in the body weight of mice during administration.

### DETAILED DESCRIPTION

The present application will be further described with reference to specific examples, and the advantages and features of the present application will become more apparent with the description. Experimental procedures without specified conditions in the examples are conducted according to conventional conditions or conditions recommended by the manufacturers. Reagents or instruments without specified manufacturers used herein are conventional products that are commercially available.

The examples of the present application are exemplary only, and do not limit the scope of the present application in any way. It will be understood by those skilled in the art that various modifications or substitutions may be made to the technical solutions of the present application in form and details without departing from the spirit and scope of the present application, and that these modifications and substitutions shall fall within the protection scope of the present application.

### TERMINOLOGY AND DEFINITIONS

Unless otherwise defined herein, scientific and technical terms used in correlation with the present application shall have the meanings that are commonly understood by those skilled in the art.

Furthermore, unless otherwise stated herein, terms used in the singular form herein shall include the plural form, and vice versa. More specifically, as used in this specification and the appended claims, unless otherwise clearly indicated, the singular forms “a”, “an”, and “the” include referents in the plural form.

The terms “include”, “comprise”, and “have” herein are used interchangeably and are intended to indicate the inclusion of a solution, implying that there may be elements other than those listed in the solution. Meanwhile, it should be understood that the descriptions “include”, “comprise”, and “have” used herein also provide the solution of “consist of”. Illustratively, “a composition, comprising A and B” should be understood as the following technical solution: a composition consisting of A and B, and a composition containing other components in addition to A and B, all of which fall within the scope of the aforementioned “a composition”.

The term “and/or” herein includes the meanings of “and”, “or”, and “all or any other combination of elements linked by the term”.

The term “MUC17” herein refers to a member of the mucin family. The mucin family includes more than 20 members. Mucins are large and highly glycosylated membrane-binding proteins that are expressed almost exclusively in the intestinal tract. Their general function is to protect epithelial cells from environmental influences, as well as to regulate the proliferation and survival of cells. MUC17 is highly expressed in pancreatic adenocarcinoma tissues. MUC17 is expressed in pancreatic cancer, appendiceal cancer, and some colon cancers. Its expression is not detected in cell lines of normal pancreas and pancreatitis or derived from other cancers.

The term “CD3” (cluster of differentiation 3) herein refers to a cluster of differentiation 3 protein derived from any vertebrate source, including mammals such as primates (e.g., humans and monkeys) and rodents (e.g., mice and rats). In mammals, the CD3 molecule is a multi-protein complex of six chains, including a CD3γ chain, a CD3δ chain, two CD3ε chains, and a homodimer of CD3ζ chains, wherein the CD3ζ chain is the intracellular tail of the CD3 molecule, and the CD3γ chain, the CD3δ chain, and the CD3P chain all contain an extracellular domain (ECD) expressed on the surface of T cells. Exemplary sequences of human CD3 include human CD3ε protein (NCBI Ref Seq No. NP_000724 or NCBI: AAH49847.1), human CD3δ protein (NCBI Ref Seq No. NP_000723), and human CD3γ protein (NCBI Ref Seq No. NP_000064). Exemplary sequences of non-human CD3 include cynomolgus monkey (Macaca fascicularis) (monkey) CD3ε protein (NCBI Ref Seq No. NP_001270544), cynomolgus monkey (Macaca fascicularis) (monkey) CD3δ protein (NCBI Ref Seq No. NP_001274617), cynomolgus monkey (Macaca fascicularis) (monkey) CD3γ protein (NCBI Ref Seq No. NP_001270839); mouse CD3ε protein (NCBI Ref Seq No. NP_031674), mouse CD3δ protein (NCBI Ref Seq No. NP_038515), mouse CD3γ protein (NCBI Ref Seq No. AAA37400); Rattus norvegicus (rat) CD3ε protein (NCBI Ref Seq No. NP_001101610), Rattus norvegicus (rat) CD3δ protein (NCBI Ref Seq No. NP_037301), and Rattus norvegicus (rat) CD3γ protein (NCBI Ref Seq No. NP_001071114).

The term “specifically bind to” herein refers to that an antigen-binding molecule (e.g., an antibody) specifically binds to an antigen and substantially identical antigens, generally with high affinity, but does not bind to unrelated antigens with high affinity. The affinity is generally reflected in an equilibrium dissociation constant (KD), where a relatively low KD indicates a relatively high affinity. In the case of antibodies, high affinity generally means having a KD of 1×10⁻⁷ M or less, about 1×10⁻⁸ M or less, about 1×10⁻⁹ M or less, about 1×10⁻¹⁰ M or less, 1×10⁻¹¹ M or less, or 1×10⁻¹² M or less. The KD is calculated as follows: KD=Kd/Ka, where Kd represents the dissociation rate and Ka represents the association rate. The equilibrium dissociation constant KD can be measured by methods well known in the art, such as surface plasmon resonance (e.g., Biacore) or equilibrium dialysis. Illustratively, KD values can be obtained by the method as described in Example 5 herein.

The term “antigen-binding molecule” herein is used in its broadest sense and refers to a molecule that specifically binds to an antigen. Illustratively, the antigen-binding molecule includes, but is not limited to, an antibody or an antibody mimetic. “Antibody mimetic” refers to an organic compound or a binding domain that is capable of specifically binding to an antigen, but is not structurally related to an antibody. Illustratively, the antibody mimetic includes, but is not limited to, affibody, affitin, affilin, a designed ankyrin repeat protein (DARPin), a nucleic acid aptamer, and a Kunitz domain peptide.

The term “antibody” herein is used in its broadest sense and refers to a polypeptide or a combination of polypeptides that comprises sufficient sequence from an immunoglobulin heavy chain variable region and/or sufficient sequence from an immunoglobulin light chain variable region to be capable of specifically binding to an antigen. “Antibody” herein encompasses various forms and various structures as long as they exhibit the desired antigen binding activity. The “antibody” herein includes alternative protein scaffolds or artificial scaffolds having grafted complementarity determining regions (CDRs) or CDR derivatives. Such scaffolds include antibody-derived scaffolds comprising mutations introduced to, for example, stabilize the three-dimensional structure of the antibody, and fully synthetic scaffolds comprising, for example, biocompatible polymers. See, e.g., Korndorfer et al., 2003, Proteins: Structure, Function, and Bioinformatics, 53(1):121-129 (2003); and Roque et al., Biotechnol. Prog. 20:639-654 (2004). Such scaffolds may also include non-antibody-derived scaffolds, such as scaffold proteins known in the art to be useful for grafting CDRs, including but not limited to, tenascin, fibronectin, peptide aptamers, and the like.

The term “antibody” herein includes a typical “four-chain antibody”, which is an immunoglobulin consisting of two heavy chains (HCs) and two light chains (LCs). The heavy chain refers to a polypeptide chain consisting of, from the N-terminus to the C-terminus, a heavy chain variable region (VH), a heavy chain constant region CH1 domain, a hinge region (HR), a heavy chain constant region CH2 domain, a heavy chain constant region CH3 domain; moreover, when the full-length antibody is of IgE isoform, the heavy chain optionally further comprises a heavy chain constant region CH4 domain. The light chain is a polypeptide chain consisting of, from the N-terminus to the C-terminus, a light chain variable region (VL) and a light chain constant region (CL). The heavy chains are connected to each other and to the light chains through disulfide bonds to form a Y-shaped structure. The heavy chain constant regions of immunoglobulins differ in their amino acid composition and arrangement, and thus in their antigenicity. Accordingly, the “immunoglobulin” herein can be divided into five classes, or isoforms of immunoglobulins, i.e., IgM, IgD, IgG, IgA, and IgE, with their corresponding heavy chains being μ, δ, γ, α, and ε chains, respectively. The Ig of the same class may also be divided into different subclasses according to the differences in the amino acid composition of the hinge regions and the number and location of disulfide bonds in the heavy chains. For example, IgG may be divided into IgG1, IgG2, IgG3, and IgG4, and IgA may be divided into IgAQ1 and IgA2. The light chains can be divided into κ or λ chains according to the differences in the constant regions. Each of the five classes of Ig may have a κ chain or a λ chain.

“Antibody” herein includes antibodies that do not comprise a light chain, e.g., heavy chain antibodies (HCAbs) produced by Camelidae species such as Camelus dromedarius, Camelus bactrianus, Lama glama, Lama guanicoe, and Vicugna pacos, as well as immunoglobulin new antigen receptors (IgNARs) found in Chondrichthyes, e.g., shark.

As used herein, the term “heavy chain antibody” refers to an antibody lacking a light chain of a conventional antibody. The term specifically includes, but is not limited to, homodimeric antibodies comprising a VH antigen-binding domain and CH2 and CH3 constant domains in the absence of a CH1 domain.

As used herein, the term “nanobody” refers to a heavy chain antibody naturally lacking a light chain present in a camel, and the cloning of its variable region can give a single domain antibody only consisting of a heavy chain variable region (also called VHH (variable domain of heavy chain of heavy chain antibody)), which is the smallest functional antigen-binding fragment.

The terms “nanobody” and “single domain antibody (sdAb)” herein have the same meaning and can be used interchangeably, and refer to a single domain antibody consisting of only one heavy chain variable region constructed by cloning a variable region of a heavy chain antibody, which is the smallest antigen-binding fragment having the complete function. Generally, a single domain antibody consisting of only one heavy chain variable region is constructed by acquiring a heavy chain antibody naturally lacking a light chain and a heavy chain constant region 1 (CH1) and then cloning the variable region of the antibody heavy chain.

For further description of “heavy chain antibody” and “nanobody”, see: Hamers-Casterman et al., Nature. 1993; 363; 446-8; a review article (Reviews in Molecular Biotechnology 74: 277-302, 2001) by Muyldermans; and the following patent applications mentioned as general background art: WO 94/04678, WO 95/04079, and WO 96/34103; WO94/25591, WO 99/37681, WO 00/40968, WO 00/43507, WO 00/65057, WO 01/40310, WO 01/44301, EP 1134231, and WO 02/48193; WO97/49805, WO 01/21817, WO 03/035694, WO 03/054016, and WO 03/055527; WO 03/050531; WO 01/90190; WO03/025020; and WO 04/041867, WO 04/041862, WO 04/041865, WO 04/041863, WO 04/062551, WO 05/044858, WO 06/40153, WO 06/079372, WO 06/122786, WO 06/122787, and WO 06/122825, as well as other prior art mentioned in these applications.

The “antibody” herein may be derived from any animal, including, but not limited to, human and non-human animals which may be selected from primates, mammals, rodents, and vertebrates, such as Camelidae species, Lama glama, Lama guanicoe, Vicugna pacos, sheep, rabbits, mice, rats, or Chondrichthyes species (e.g., shark).

The term “multispecific” herein means having at least two antigen-binding sites, each of which binds to a different epitope of the same antigen or a different epitope of a different antigen. Thus, the terms such as “bispecific”, “trispecific”, and “tetraspecific” refer to the number of different epitopes to which an antibody/antigen-binding molecule can bind.

The term “valent” herein refers to the presence of a specified number of binding sites in an antibody/antigen-binding molecule. Thus, the terms “monovalent”, “divalent”, “tetravalent”, and “hexavalent” refer to the presence of one binding site, two binding sites, four binding sites, and six binding sites, respectively, in an antibody/antigen-binding molecule.

The terms “antigen-binding fragment” and “antibody fragment” herein are used interchangeably and refer to a fragment that does not have the entire structure of an intact antibody, but comprises only a portion of the intact antibody or a variant of the portion that retains the ability to bind to an antigen. “Antigen-binding fragment” or “antibody fragment” herein includes but is not limited to, a Fab, a Fab′, a Fab′-SH, a F(ab′)₂, an Fd, an Fv, an scFv, a diabody, and a single domain antibody.

An intact antibody is digested by papain to produce two identical antigen-binding fragments, called “Fab” fragments, each of which contains a heavy chain variable domain and a light chain variable domain, as well as a light chain constant domain and a first heavy chain constant domain (CH1). Thus, the term “Fab fragment” herein refers to an antibody fragment comprising a light chain fragment comprising the VL domain and the constant domain (CL) of a light chain, and the VH domain and the first constant domain (CH1) of a heavy chain. The Fab′ fragment differs from the Fab fragment by the addition of a few residues (including one or more cysteines from an antibody hinge region) at the carboxyl terminus of the heavy chain CH1 domain. Fab′-SH is a Fab′ fragment in which the cysteine residue in the constant domain carries a free thiol group. Pepsin treatment produces an F(ab′)2 fragment having two antigen-binding sites (two Fab fragments) and a portion of the Fc region.

The term “Fd” herein refers to an antibody consisting of VH and CH1 domains. The term “Fv” herein refers to an antibody fragment consisting of VL and VH domains of a single arm. An Fv fragment is generally considered to be the smallest antibody fragment that can form an intact antigen-binding site.

It is generally believed that the six CDRs provide antigen-binding specificity to the antibody. However, even one variable region (e.g., an Fd fragment, which contains only three CDRs specific to an antigen) is capable of recognizing and binding to an antigen, although its affinity may be lower than an intact binding site.

The term “scFv” (single-chain variable fragment) herein refers to a single polypeptide chain comprising VL and VH domains, wherein the VL and VH are linked through a linker (see, e.g., Bird et al., Science 242: 423-426 (1988); Huston et al., Proc. Natl. Acad. Sci. USA 85: 5879-5883 (1988); and Pluckthun, The Pharmacology of Monoclonal Antibodies, Vol. 113, Roseburg and Moore Ed., Springer-Verlag, New York, pp 269-315 (1994)). Such scFv molecules may have a general structure: NH₂-VL-linker-VH-COOH or NH₂-VH-linker-VL-COOH. An appropriate linker in the prior art consists of GGGGS amino acid sequence repeats or a variant thereof. For example, a linker having the amino acid sequence (GGGGS)4 can be used, and variants thereof can also be used (Holliger et al. (1993), Proc. Natl. Acad. Sci. USA 90: 6444-6448). Other linkers that can be used in the present application are described in Alfthan et al. (1995), Protein Eng. 8: 725-731; Choi et al. (2001), Eur J. Immunol. 31: 94-106; Hu et al. (1996), Cancer Res. 56: 3055-3061; Kipriyanov et al. (1999), J. Mol. Biol. 293: 41-56; and Roovers et al. (2001), Cancer Immunol. In some cases, there may also be disulfide bonds between the VH and VL of the scFv, forming a disulfide-linked Fv (dsFv).

The term “diabody” herein refers to an antibody having VH and VL domains that are expressed on a single polypeptide chain, but using a linker that is too short to allow the pairing of the two domains on the same chain, thereby forcing the domains to pair with the complementary domains of the other chain and generating two antigen-binding sites (see, e.g., Holliger P. et al., Proc. Natl. Acad. Sci. USA 90: 6444-6448 (1993), and Poljak R. J. et al., Structure 2: 1121-1123 (1994)).

The term “naked antibody” herein refers to an antibody that is not conjugated to a therapeutic agent or tracer. The term “conjugated antibody” herein refers to an antibody conjugated to a therapeutic agent or tracer.

The term “humanized antibody” herein refers to a genetically engineered non-human antibody that has an amino acid sequence modified to increase homology to the sequence of a human antibody. Generally, all or part of the CDRs of a humanized antibody are derived from a non-human antibody (donor antibody), and all or part of the non-CDRs (e.g., variable region FRs and/or constant regions) are derived from a human immunoglobulin (receptor antibody). The humanized antibody generally retains or partially retains the desired properties of the donor antibody, including, but not limited to, antigen specificity, affinity, reactivity, the ability to increase the activity of immune cells, the ability to enhance immune response, and the like.

The term “fully human antibody” herein refers to an antibody having variable regions in which both the FRs and CDRs are derived from human germline immunoglobulin sequences. Furthermore, if the antibody comprises constant regions, the constant regions are also derived from human germline immunoglobulin sequences. The fully human antibody herein may include amino acid residues that are not encoded by human germline immunoglobulin sequences (e.g., mutations introduced by random or site-specific mutagenesis in vitro or by somatic mutations in vivo). However, “fully human antibody” herein does not include antibodies in which CDR sequences derived from the germline of another mammalian species (e.g., mouse) have been grafted onto human framework sequences.

The term “variable region” herein refers to a region of a heavy or light chain of an antibody involved in the binding of the antibody to an antigen. The “heavy chain variable region” is used interchangeably with “VH” and “HCVR”, and the “light chain variable region” is used interchangeably with “VL” and “LCVR”. Heavy and light chain variable domains (VH and VL, respectively) of natural antibodies generally have similar structures, each of which contains four conservative framework regions (FRs) and three hypervariable regions (HVRs). See, e.g., Kindt et al., Kuby Immunology, 6th ed., W. H. Freeman and Co., p. 91 (2007). A single VH or VL domain may be sufficient to provide antigen binding specificity. The terms “complementarity determining region” and “CDR” herein are used interchangeably and generally refer to a hypervariable region (HVR) of a heavy chain variable region (VH) or a light chain variable region (VL), which is also known as the complementarity determining region as it is precisely complementary to an epitope in spatial structures, wherein the heavy chain variable region CDR may be abbreviated as HCDR and the light chain variable region CDR may be abbreviated as LCDR. The terms “framework region” or “FR” are used interchangeably and refer to those amino acid residues of an antibody heavy chain variable region or light chain variable region other than CDRs. Generally, a typical antibody variable region consists of 4 FRs and 3 CDRs in the following order: FR1-CDR1-FR2-CDR2-FR3-CDR3-FR4.

For further description of the CDRs, see Kabat et al., J. Biol. Chem., 252: 6609-6616 (1977); Kabat et al., United States Department of Health and Human Services, Sequences of proteins of immunological interest (1991); Chothia et al., J. Mol. Biol. 196: 901-917 (1987); Al-Lazikani B. et al., J. Mol. Biol., 273: 927-948 (1997); MacCallum et al., J. Mol. Biol. 262: 732-745 (1996); Abhinandan and Martin, Mol. Immunol., 45: 3832-3839 (2008); Lefranc M. P. et al., Dev. Comp. Immunol., 27: 55-77 (2003); and Honegger and Plückthun, J Mol. Biol., 309: 657-670 (2001). The “CDR” herein may be labeled and defined in a manner well known in the art, including but not limited to Kabat numbering scheme, Chothia numbering scheme, or IMGT numbering scheme; the tool websites used include, but are not limited to, AbRSA site (http://cao.labshare.cn/AbRSA/cdrs.php), abYsis site (www.abysis.org/abysis/sequence_input/key_annotation/key_annotation.cgi), and IMGT site (http://www.imgt.org/3Dstructure-DB/cgi/DomainGapAlign.cgi#results). The CDR herein includes overlaps and subsets of amino acid residues defined in different ways.

The term “Kabat numbering scheme” herein generally refers to the immunoglobulin alignment and numbering scheme proposed by Elvin A. Kabat (see, e.g., Kabat et al., Sequences of Proteins of Immunological Interest, 5th Ed. Public Health Service, National Institutes of Health, Bethesda, Md., 1991).

The term “IMGT numbering scheme” herein generally refers to a numbering scheme based on the international ImMunoGeneTics information system (IMGT) initiated by Lefranc et al., see Lefranc et al., Dev. Comparat. Immunol. 27: 55-77, 2003.

The term “Chothia numbering scheme” herein generally refers to the immunoglobulin numbering scheme proposed by Chothia et al., which is a classical rule for identifying CDR region boundaries based on the position of structural loop regions (see, e.g., Chothia & Lesk (1987) J Mol. Biol. 196: 901-917; Chothia et al., (1989) Nature 342: 878-883).

The term “heavy chain constant region” herein refers to the carboxyl-terminal portion of an antibody heavy chain that is not directly involved in the binding of the antibody to an antigen, but exhibits effector functions, such as interaction with an Fc receptor; the heavy chain constant region has a more conserved amino acid sequence relative to the variable domain of the antibody. The “heavy chain constant region” at least comprises: a CH1 domain, a hinge region, a CH2 domain, a CH3 domain, or a variant or fragment thereof. The “heavy chain constant region” includes a “full-length heavy chain constant region” having a structure substantially similar to that of a natural antibody constant region and a “heavy chain constant region fragment” including only “a portion of the full-length heavy chain constant region”. Illustratively, a typical “full-length antibody heavy chain constant region” consists of the CH1 domain-hinge region-CH2 domain-CH3 domain. When the antibody is IgE, it further comprises a CH4 domain; when the antibody is a heavy chain antibody, it does not comprise a CH1 domain. Illustratively, a typical “heavy chain constant region fragment” may be selected from CH1, Fc, and CH3 domains.

The term “light chain constant region” herein refers to the carboxyl-terminal portion of an antibody light chain that is not directly involved in the binding of the antibody to an antigen. The light chain constant region may be selected from a constant κ domain and a constant λ domain.

The term “Fc” herein refers to the carboxyl-terminal portion of an antibody that is formed by the hydrolysis of an intact antibody by papain, which typically comprises the CH3 and CH2 domains of the antibody. The Fc region includes, for example, an Fc region of native sequences, a recombinant Fc region, and a variant Fc region. Although the boundaries of the Fc region of an immunoglobulin heavy chain may vary slightly, the human IgG heavy chain Fc region is generally defined as extending from an amino acid residue at position Cys226, or from Pro230, to the carboxyl terminus thereof. The C-terminal lysine of the Fc region (residue 447 according to the Kabat numbering scheme) may be removed, for example, during production or purification of the antibody, or by recombinant engineering of the nucleic acid encoding the heavy chain of the antibody, and thus, the Fc region may or may not include Lys447.

The term “conservative amino acid” herein generally refers to amino acids that belong to the same class or have similar characteristics (e.g., charge, side chain size, hydrophobicity, hydrophilicity, backbone conformation, and rigidity). Illustratively, the amino acids in each of the following groups belong to conservative amino acid residues of each other, and substitutions of amino acid residues within the groups belong to conservative amino acid substitutions:

Illustratively, the following six groups are examples of amino acids that are considered to be conservative replacements of each other:
1) alanine (A), serine (S), and threonine (T);
2) aspartic acid (D) and glutamic acid (E);
3) asparagine (N) and glutamine (Q);
4) arginine (R), lysine (K), and histidine (H);
5) isoleucine (I), leucine (L), methionine (M), and valine (V); and
6) phenylalanine (F), tyrosine (Y), and tryptophan (W).

The term “identity” herein can be obtained by calculating as follows: to determine the percent “identity” of two amino acid sequences or two nucleic acid sequences, the sequences are aligned for optimal comparison purposes (e.g., for optimal alignment, gaps can be introduced in one or both of the first and second amino acid sequences or nucleic acid sequences, or non-homologous sequences can be discarded for comparison). Amino acid residues or nucleotides at corresponding amino acid positions or nucleotide positions are then compared. When a position in the first sequence is occupied by the same amino acid residue or nucleotide at the corresponding position in the second sequence, the molecules are identical at this position.

The percent identity between two sequences varies with the identical positions shared by the sequences, taking into account the number of gaps that need to be introduced and the length of each gap for optimal alignment of the two sequences.

A mathematical algorithm can be used to compare two sequences and calculate the percent identity between the sequences. For example, the percent identity between two amino acid sequences is determined with the Needlema and Wunsch algorithm ((1970) J. Mol. Biol., 48: 444-453; available at www.gcg.com) which has been integrated into the GAP program of the GCG software package, using the Blossum 62 matrix or PAM250 matrix and gap weight of 16, 14, 12, 10, 8, 6, or 4 and length weight of 1, 2, 3, 4, 5, or 6. For another example, the percent identity between two nucleotide sequences is determined with the GAP program of the GCG software package (available at www.gcg.com), using the NWSgapdna.CMP matrix and gap weight of 40, 50, 60, 70, or 80 and length weight of 1, 2, 3, 4, 5, or 6. A particularly preferred parameter set (and one that should be used unless otherwise stated) is a Blossum62 scoring matrix with a gap penalty of 12, a gap extension penalty of 4, and a frameshift gap penalty of 5.

The percent identity between two amino acid sequences or nucleotide sequences can also be determined with a PAM120 weighted remainder table, a gap length penalty of 12, and a gap penalty of 4, using the E. Meyers and W. Miller algorithm ((1989) CABIOS, 4: 11-17) which has been incorporated into the ALIGN program (version 2.0).

Additionally or alternatively, the nucleic acid sequences and protein sequences described herein can be further used as “query sequences” to perform searches against public databases to, e.g., identify other family member sequences or correlated sequences. For example, such searches can be performed using the NBLAST and XBLAST programs (version 2.0) of Altschul et al., (1990) J. Mol. Biol., 215: 403-10. BLAST nucleotide searches can be performed using the NBLAST program, with a score of 100 and a word length of 12, to obtain nucleotide sequences homologous to the nucleic acid (SEQ ID NO: 1) molecule of the present application. BLAST protein searches can be performed using the XBLAST program, with a score of 50 and a word length of 3, to acquire amino acid sequences homologous to the protein molecule of the present application. To obtain gapped alignment results for the purpose of comparison, gapped BLAST can be used as described in Altschul et al. (1997) Nucleic Acids Res. 25: 3389-3402. When using the BLAST and gapped BLAST programs, the default parameters of the respective programs (e.g., XBLAST and NBLAST) can be used. See www.ncbi.nlm.nih.gov.

The term “nucleic acid” herein includes any compound and/or substance that comprises a polymer of nucleotides. Each nucleotide consists of a base, in particular a purine or pyrimidine base (i.e., cytosine (C), guanine (G), adenine (A), thymine (T), or uracil (U)), a sugar (i.e., deoxyribose or ribose), and a phosphate group. Generally, a nucleic acid molecule is described as a sequence of bases, whereby the bases represent the primary structure (linear structure) of the nucleic acid molecule. The sequence of bases is generally expressed as 5′ to 3′. Herein, the term “nucleic acid molecule” encompasses deoxyribonucleic acid (DNA), including, e.g., complementary DNA (cDNA) and genomic DNA; ribonucleic acid (RNA), in particular messenger RNA (mRNA); the synthetic forms of DNA or RNA; and polymers comprising a mixture of two or more of these molecules. The nucleic acid molecule may be linear or cyclic. Furthermore, the term “nucleic acid molecule” includes both sense and antisense strands, as well as single- and double-stranded forms. Moreover, the nucleic acid molecules described herein may contain naturally occurring or non-naturally occurring nucleotides. Examples of non-naturally occurring nucleotides include modified nucleotide bases having derived sugar or phosphate backbone linkages or chemically modified residues. The nucleic acid molecule also encompasses DNA and RNA molecules suitable for use as vectors for direct expression of the antibodies of the present application in vitro and/or in vivo, e.g., in a host or patient. Such DNA (e.g., cDNA) or RNA (e.g., mRNA) vectors may be unmodified or modified. For example, mRNA may be chemically modified to enhance the stability of the RNA vector and/or the expression of the encoded molecule, so that the mRNA can be injected into a subject to produce antibodies in vivo (see, e.g., Stadler et al., Nature Medicine 2017, published online, Jun. 12, 2017, doi: 10.1038/nm.4356 or EP 2 101 823 B1). The “Isolated” nucleic acid herein refers to a nucleic acid molecule that has been separated from components of its natural environment. The isolated nucleic acid includes a nucleic acid molecule contained in such a cell: the cell generally contains the nucleic acid molecule, but the nucleic acid molecule is present extrachromosomally or at a chromosomal location different from its natural chromosomal location.

The term “vector” herein refers to a nucleic acid molecule capable of amplifying another nucleic acid to which it has been linked. The term includes vectors that serve as self-replicating nucleic acid structures as well as vectors integrated into the genome of a host cell into which they have been introduced. Certain vectors are capable of directing the expression of nucleic acids to which they are operably linked. Such vectors are called “expression vectors” herein.

The term “host cell” herein refers to a cell into which an exogenous nucleic acid has been introduced, including the progeny of such a cell. Host cells include “transformants” and “transformed cells”, which include primary transformed cells and progenies derived therefrom, regardless of the number of passages. Progenies may not be exactly the same as parent cells in terms of nucleic acid content, and may contain mutations. Mutant progenies having the same function or biological activity that are screened or selected from the primary transformed cells are included herein.

The term “pharmaceutical composition” herein refers to a formulation that exists in a form allowing the biological activity of the active ingredient contained therein to be effective and does not contain additional ingredients having unacceptable toxicity to a subject to which the pharmaceutical composition is administered. The “pharmaceutical composition” may further comprise an additional therapeutic agent, such as an antineoplastic agent; more preferably, the antineoplastic agent may be specifically a PD-1 axis binding antagonist, a small molecule antineoplastic agent, or a cell therapeutic agent, and the cell therapeutic agent may be CAR-T, CAR-NK, etc.

The term “subject” herein refers to an organism that receives treatment for a particular disease or disorder described herein. Examples of subjects and patients include mammals, such as humans, primates (e.g., monkeys), or non-primate mammals, that receive treatment for a disease or disorder.

The term “treatment” herein refers to surgical or therapeutic treatment for the purpose of preventing or slowing (reducing) the progression of an undesired physiological or pathological change, e.g., a cancer, in a subject being treated. Beneficial or desired clinical results include, but are not limited to, alleviation of symptoms, decrease of severity of disease, stabilization (i.e., not worsening) of state of disease, delay or slowing of disease progression, amelioration or palliation of state of disease, and remission of state of disease (whether partial or total), whether detectable or undetectable. Subjects in need of treatment include those already with a disorder or disease, as well as those who are susceptible to a disorder or disease or those who intend to prevent a disorder or disease. When referring to terms such as slowing, alleviation, decrease, palliation, and remission, their meanings also include elimination, disappearance, nonoccurrence, etc.

The term “immunostimulatory antibody” herein can promote anti-tumor immunity by directly modulating immune functions, i.e., blocking other inhibitory targets or enhancing immunostimulatory proteins. Immunostimulatory antibodies include 1) antagonistic antibodies targeting inhibitory immune checkpoints and agonistic antibodies for enhancing immunostimulatory proteins.

The term “immunomodulatory drug” herein may be, for example, thymosin al. Principle: Thymosin α1 (Tα1) is a naturally occurring thymus peptide that acts as an endogenous regulator of the innate and adaptive immune systems. It is used worldwide for treating diseases associated with immune dysfunction, including viral infections such as hepatitis B and C and certain cancers, and for vaccine enhancement. In particular, recent advances in immunomodulation studies point to a beneficial effect of Tal treatment in septic patients (Wu et al. Critical Care 2013, 17:R8).

The term “effective amount” herein refers to an amount of a therapeutic agent that is effective to prevent or alleviate symptoms of a disease or the progression of the disease when administered to a cell, tissue, or subject alone or in combination with another therapeutic agent. “Effective amount” also refers to an amount of a compound that is sufficient to alleviate symptoms, e.g., to treat, cure, prevent, or alleviate related medical disorders, or to increase the rates at which such disorders are treated, cured, prevented, or alleviated. When the active ingredient is administered alone to an individual, a therapeutically effective dose refers to the amount of the ingredient alone. When a combination is used, a therapeutically effective dose refers to the combined amounts of the active ingredients that produce the therapeutic effect, whether administered in combination, sequentially, or simultaneously.

The term “cancer” herein refers to or describes a physiological condition in mammals that is typically characterized by unregulated cell growth. Included in this definition are benign and malignant cancers.

The term “tumor” or “neoplasm” herein refers to all neoplastic cell growth and proliferation, whether malignant or benign, and all pre-cancerous and cancerous cells and tissues. The terms “cancer” and “tumor” are not mutually exclusive when referred to herein.

The term “EC50” herein refers to the half maximum effective concentration, which includes the antibody concentration that induces a halfway response between the baseline and maximum after a specified exposure time. EC50 essentially represents the antibody concentration at which 50% of the maximal effect is observed, and can be measured by methods known in the art.

### EXAMPLES

### Example 1: Design and Construction of Bispecific Antibodies

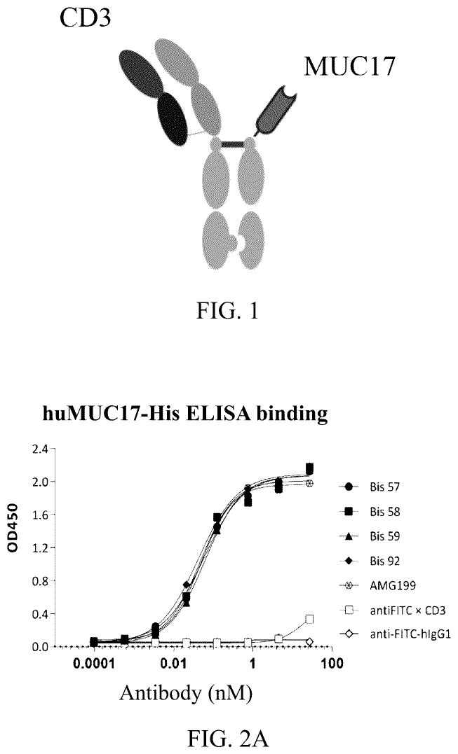

The anti-MUC17 humanized nanobody sequences and the anti-CD3 humanized antibody sequences were used to construct bispecific antibody molecules targeting MUC17×CD3, wherein the MUC17 variable region sequences were derived from the patent PCT/CN2022/126410, and the variable region sequences of the CD3 antibody were derived from the patent PCT/CN2022/118334. The bispecific antibody comprises three chains, that is, a heavy chain comprising the VH of the anti-CD3 humanized antibody, a light chain comprising the VL of the anti-CD3 humanized antibody, and a heavy chain of the anti-MUC17 humanized nanobody. The structure of the bispecific antibody is shown in FIG. 1. To reduce homologous mispairing, an asymmetric structure was adopted in the molecule, and KIH mutations were introduced into the Fc regions of the two heavy chains. Meanwhile, to reduce the ADCC function and CDC function of the antibody and avoid damage to T cells and potential toxic and side effects, mutations L234A, L235A, and G237A were introduced into the heavy chain constant regions. The constructed bispecific antibodies were named Bis 57, Bis 58, Bis 59, and Bis 92, and the amino acid sequence of each chain is shown in Table 1 below. The sequence of the control antibody AMNG199 was derived from the published patent WO2019133961. The specific sequences are shown in Table 1.

TABLE 1
Amino acid sequences of bispecific antibodies

| | Antibody | |
|---|---|---|
| BsAb name | Sequence Name | Amino acid sequence |
| Bis 57 | Knob chain SEQ ID NO. 1 | 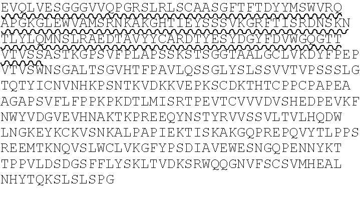 |
| | | Note: CD3 VH (wavy line) |
| | LC light chain SEQ ID NO. 2 | 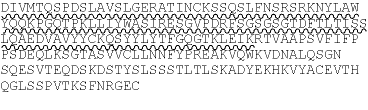 |
| | | Note: CD3 VL (wavy line) |
| | Hole chain | EVQLVESGGGLVQPGRSLRLSCAASGDITRRCMGWFRQAP |
| | SEQ ID NO. 3 | GKEREGVAGISEDGITIYADSVKGRFTISKDNAKNSLYLQM |
| | | NSLRAEDTALYSCAASILNRVGCRQLSYEYIYRGQGTMVT |
| | | VSSGGGDKTHTCPPCPAPEAAGAPSVFLFPPKPKDTLMISR |
| | | TPEVTCVVVDVSHEDPEVKFNWYVDGVEVHNAKTKPREE |
| | | QYNSTYRVVSVLTVLHQDWLNGKEYKCKVSNKALPAPIE |
| | | KTISKAKGQPREPQVYTLPPSREEMTKNQVSLSCAVKGFYP |
| | | SDIAVEWESNGQPENNYKTTPPVLDSDGSFFLVSKLTVDKS |
| | | RWQQGNVFSCSVMHEALHNHYTQKSLSLSPG |
| | | Note: MUC17 VHH 7724H5 (single underline) |
| Bis 59 | Knob chain | SEQ ID NO. 1 |
| | LC light chain | SEQ ID NO. 2 |
| | Hole chain | EVQLVESGGGVVQPGGSLRLSCAASGDTFSRGSWGWFRQ |
| | SEQ ID NO. 4 | APGKEREAVALISSDGWTFYADSVKGRFTISQDNSKNTLYL |
| | | QMNSLRAEDTAVYYCAASVLVLRTLNPKAWHHWGQGTM |
| | | VTVSSGGGDKTHTCPPCPAPEAAGAPSVFLFPPKPKDTLMI |
| | | SRTPEVTCVVVDVSHEDPEVKFNWYVDGVEVHNAKTKPR |
| | | EEQYNSTYRVVSVLTVLHQDWLNGKEYKCKVSNKALPAPI |
| | | EKTISKAKGQPREPQVYTLPPSREEMTKNQVSLSCAVKGF |
| | | YPSDIAVEWESNGQPENNYKTTPPVLDSDGSFFLVSKLTVD |
| | | KSRWQQGNVFSCSVMHEALHNHYTQKSLSLSPG |
| | | Note: MUC17 VHH 7785H3 (single underline) |
| Bis 58 | Knob chain | SEQ ID NO. 1 |
| | LC light chain | SEQ ID NO. 2 |
| | Hole chain | EVQLVESGGGLVQPGGSLRLSCAASGFPSSRGTMAWFRQA |
| | SEQ ID NO. 5 | PGKEREGVAFINSDGRTSYAESVKGRFTISRDNAKNSLYLQ |
| | | MNSLRAEDTAVYYCAASLLYLMTVNAAAYPYWGQGTMV |
| | | TVSSGGGDKTHTCPPCPAPEAAGAPSVFLFPPKPKDTLMIS |
| | | RTPEVTCVVVDVSHEDPEVKFNWYVDGVEVHNAKTKPRE |
| | | EQYNSTYRVVSVLTVLHQDWLNGKEYKCKVSNKALPAPIE |
| | | KTISKAKGQPREPQVYTLPPSREEMTKNQVSLSCAVKGFYP |
| | | SDIAVEWESNGQPENNYKTTPPVLDSDGSFFLVSKLTVDKS |
| | | RWQQGNVFSCSVMHEALHNHYTQKSLSLSPG |
| | | Note: MUC17 VHH 7783H2 (single underline) |
| Bis 92 | Knob chain | SEQ ID NO. 1 |
| | LC light chain | SEQ ID NO. 2 |
| | Hole chain | EVQLVESGGGVVQPGGSLRLSCAASGYTSNRYSMAWFRQ |
| | SEQ ID NO. 6 | APGKEREVVAFIWTDGGRTYYADSVKGRFTISRDNSKNTL |
| | | YLQMNSLRAEDTAVYYCAASSLLLQTINPRAWPYWGQGT |
| | | MVTVSSGGGDKTHTCPPCPAPEAAGAPSVFLFPPKPKDTL |
| | | MISRTPEVTCVVVDVSHEDPEVKFNWYVDGVEVHNAKTK |
| | | PREEQYNSTYRVVSVLTVLHQDWLNGKEYKCKVSNKALP |
| | | APIEKTISKAKGQPREPQVYTLPPSREEMTKNQVSLSCAVK |
| | | GFYPSDIAVEWESNGQPENNYKTTPPVLDSDGSFFLVSKLT |
| | | VDKSRWQQGNVFSCSVMHEALHNHYTQKSLSLSPG |
| | | Note: MUC17 VHH 7784H1 (single underline) |
| antiFITCx CD3 | Knob chain | SEQ ID NO. 1 |
| negative control | light chain | SEQ ID NO. 2 |
| | Hole chain | EVKLDETGGGLVQPGRPMKLSCVASGFTFSDYWMNWVR |
| | SEQ ID NO. 7 | QSPEKGLEWVAQIRNKPYNYETYYSDSVKGRFTISRDDSK |
| | | SSVYLQMNNLRVEDMGIYYCTGSYYGMDYWGQGTSVTV |
| | | SSGGGGSGGGGSGGGGSDVVMTQTPLSLPVSLGDQASISC |
| | | RSSQSLVHSNGNTYLRWYLQKPGQSPKVLIYKVSNRFSGV |
| | | PDRFSGSGSGTDFTLKISRVEAEDLGVYFCSQSTHVPWTFG |
| | | GGTKLEIKGGGDKTHTCPPCPAPEAAGAPSVFLFPPKPKDT |
| | | LMISRTPEVTCVVVDVSHEDPEVKFNWYVDGVEVHNAKT |
| | | KPREEQYNSTYRVVSVLTVLHQDWLNGKEYKCKVSNKAL |
| | | PAPIEKTISKAKGQPREPQVYTLPPSREEMTKNQVSLSCAV |
| | | KGFYPSDIAVEWESNGQPENNYKTTPPVLDSDGSFFLVSKL |
| | | TVDKSRWQQGNVFSCSVMHEALHNHYTQKSLSLSPG |
| | | Note: antiFITC-scFV (single underline) |
| AMG199 | SEQ ID NO. 8 | QVQLQQWGAGLLKPSETLSLTCAVYGGSFSGYYWSWIRQ |
| | | PPGKCLEWIGDIDASGSTKYNPSLKSRVTISLDTSKNQFSLK |
| | | LNSVTAADTAVYFCARKKYSTVWSYFDNWGQGTLVTVSS |
| | | GGGGSGGGGSGGGGSSYELTQPSSVSVPPGQTASITCSGDK |
| | | LGDKYASWYQQKPGQSPVLVIYQDRKRPSGVPERFSGSNS |
| | | GNTATLTISGTQAMDEADYYCQAWGSSTAVFGCGTKLTVL |
| | | SGGGSEVQLVESGGGLVQPGGSLKLSCAASGFTFNKYAM |
| | | NWVRQAPGKGLEWVARIRSKYNNYATYYADSVKDRFTISR |
| | | DDSKNTAYLQMNNLKTEDTAVYYCVRHGNFGNSYISYWA |
| | | YWGQGTLVTVSSGGGGSGGGGSGGGGSQTVVTQEPSLTV |
| | | SPGGTVTLTCGSSTGAVTSGNYPNWVQQKPGQAPRGLIGG |
| | | TKFLAPGTPARFSGSLLGGKAALTLSGVQPEDEAEYYCVL |
| | | WYSSNRWVFGGGTKLTVLGGGGDKTHTCPPCPAPELLGGP |
| | | SVFLFPPKPKDTLMISRTPEVTCVVVDVSHEDPEVKFNWY |
| | | VDGVEVHNAKTKPCEEQYGSTYRCVSVLTVLHQDWLNG |
| | | KEYKCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRWQQGNVFSCSVMHEALHNHY |
| | | TQKSLSLSPGKGGGGSGGGGSGGGGSGGGGSGGGGSGGG |
| | | GSDKTHTCPPCPAPELLGGPSVFLFPPKPKDTLMISRTPEVT |
| | | CVVVDVSHEDPEVKFNWYVDGVEVHNAKTKPCEEQYGS |
| | | TYRCVSVLTVLHQDWLNGKEYKCKVSNKALPAPIEKTISK |
| | | AKGQPREPQVYTLPPSREEMTKNQVSLTCLVKGFYPSDIAV |
| | | EWESNGQPENNYKTTPPVLDSDGSFFLYSKLTVDKSRWQQ |
| | | GNVFSCSVMHEALHNHYTQKSLSLSPGK |

TABLE 2
Variable region sequences of bispecific antibodies

| Variable | | |
|---|---|---|
| region | Sequence No. | sequence |
| CD3 VH | SEQ ID NO. 9 | EVQLVESGGGVVQPGRSLRLSCAASGFTFTDYYMSWVRQA |
| | | PGKGLEWVAMSRNKAKGHTIEYSSSVKGRFTISRDNSKNT |
| | | LYLQMNSLRAEDTAVYYCARDTYESYDGYFDVWGQGTTVT |
| | | VSS |
| CD3 VL | SEQ ID NO. 10 | DIVMTQSPDSLAVSLGERATINCKSSQSLFNSRSRKNYLA |
| | | WYQQKPGQTPKLLIYWASIRESGVPDRFSGSGSGTDFTLT |
| | | ISSLQAEDVAVYYCKQSYYLYTFGQGTKLEIK |
| 7724H5 | SEQ ID NO. 11 | EVQLVESGGGLVQPGRSLRLSCAASGDITRRCMGWFRQAP |
| | | GKEREGVAGISEDGITIYADSVKGRFTISKDNAKNSLYLQ |
| | | MNSLRAEDTALYSCAASILNRVGCRQLSYEYIYRGQGTMV |
| | | TVSS |
| 7785H3 | SEQ ID NO. 12 | EVQLVESGGGVVQPGGSLRLSCAASGDTFSRGSWGWFRQA |
| | | PGKEREAVALISSDGWTFYADSVKGRFTISQDNSKNTLYL |
| | | QMNSLRAEDTAVYYCAASVLVLRTLNPKAWHHWGQGTMVT |
| | | VSS |
| 7783H2 | SEQ ID NO. 13 | EVQLVESGGGLVQPGGSLRLSCAASGFPSSRGTMAWFRQA |
| | | PGKEREGVAFINSDGRTSYAESVKGRFTISRDNAKNSLYL |
| | | QMNSLRAEDTAVYYCAASLLYLMTVNAAAYPYWGQGTMVT |
| | | VSS |
| 7784H1 | SEQ ID NO. 14 | EVQLVESGGGVVQPGGSLRLSCAASGYTSNRYSMAWFRQA |
| | | PGKEREVVAFIWTDGGRTYYADSVKGRFTISRDNSKNTLY |
| | | LQMNSLRAEDTAVYYCAASSLLLQTINPRAWPYWGQGTMV |
| | | TVSS |

TABLE 3
CDR analysis results for bispecific antibodies
Kabat analysis results

| Variable | | | |
|---|---|---|---|
| region | HCDR1 | HCDR2 | HCDR3 |
| CD3 VH | DYYMS | MSRNKAKGHTIEY | DTYESYDGYFDV |
| | SEQ ID NO. 15 | SSSVKG | SEQ ID NO. 17 |
| | | SEQ ID NO. 16 | |
| Variable | | | |
| region | LCDR1 | LCDR2 | LCDR3 |
| CD3 VL | KSSQSLFNSRSRK | WASIRES | KQSYYLYT |
| | NYLA | SEQ ID NO. 19 | SEQ ID NO. 20 |
| | SEQ ID NO. 18 | | |
| Variable | | | |
| region | CDR1 | CDR2 | CDR3 |
| 7724H5 | RCMG | GISEDGITIYADS | SILNRVGCRQLSY |
| | SEQ ID NO. 21 | VKG | EYIY |
| | | SEQ ID NO. 22 | SEQ ID NO. 23 |
| 7785H3 | RGSWG | LISSDGWTFYADS | SVLVLRTLNPKAW |
| | SEQ ID NO. 24 | VKG | HH |
| | | SEQ ID NO. 25 | SEQ ID NO. 26 |
| 7783H2 | RGTMA | FINSDGRTSYAES | SLLYLMTVNAAAY |
| | SEQ ID NO. 27 | VKG | PY |
| | | SEQ ID NO. 28 | SEQ ID NO. 29 |
| 7784H1 | RYSMA | FIWTDGGRTYYAD | SSLLLQTINPRAW |
| | SEQ ID NO. 30 | SVKG | PY |
| | | SEQ ID NO. 31 | SEQ ID NO. 32 |

Chothia analysis results

| Variable | | | |
|---|---|---|---|
| region | HCDR1 | HCDR2 | HCDR3 |
| CD3 VH | GFTFTDY | RNKAKGHT | DTYESYDGYFDV |
| | SEQ ID NO. 33 | SEQ ID NO. 34 | SEQ ID NO. 35 |
| Variable | | | |
| region | LCDR1 | LCDR2 | LCDR3 |
| CD3 VL | KSSQSLFNSRSRK | WASIRES | KQSYYLYT |
| | NYLA | SEQ ID NO. 37 | SEQ ID NO. 38 |
| | SEQ ID NO. 36 | | |
| Variable | | | |
| region | CDR1 | CDR2 | CDR3 |
| 7724H5 | GDITRR | SEDGI | SILNRVGCRQLSY |
| | SEQ ID NO. 39 | SEQ ID NO. 40 | EYIY |
| | | | SEQ ID NO. 41 |
| 7785H3 | GDTFSRG | SSDGW | SVLVLRTLNPKAW |
| | SEQ ID NO. 42 | SEQ ID NO. 43 | HH |
| | | | SEQ ID NO. 44 |
| 7783H2 | GFPSSRG | NSDGR | SLLYLMTVNAAAY |
| | SEQ ID NO. 45 | SEQ ID NO. 46 | PY |
| | | | SEQ ID NO. 47 |
| 7784H1 | GYTSNRY | WTDGGR | SSLLLQTINPRAW |
| | SEQ ID NO. 48 | SEQ ID NO. 49 | PY |
| | | | SEQ ID NO. 50 |

IMGT analysis results

| Variable | | | |
|---|---|---|---|
| region | HCDR1 | HCDR2 | HCDR3 |
| CD3 VH | GFTFTDYY | SRNKAKGHTI | ARDTYESYDGYFD |
| | SEQ ID NO. 51 | SEQ ID NO. 52 | V |
| | | | SEQ ID NO. 53 |
| Variable | | | |
| region | LCDR1 | LCDR2 | LCDR3 |
| CD3 VL | QSLFNSRSRKNY | WAS | KQSYYLYT |
| | SEQ ID NO. 54 | SEQ ID NO. 55 | SEQ ID NO. 56 |
| Variable | | | |
| region | CDR1 | CDR2 | CDR3 |
| 7724H5 | GDITRRC | ISEDGIT | AASILNRVGCRQL |
| | SEQ ID NO. 57 | SEQ ID NO. 58 | SYEYIY |
| | | | SEQ ID NO. 59 |
| 7785H3 | GDTFSRGS | ISSDGWT | AASVLVLRTLNPK |
| | SEQ ID NO. 60 | SEQ ID NO. 61 | AWHH |
| | | | SEQ ID NO. 62 |
| 7783H2 | GFPSSRGT | INSDGRT | AASLLYLMTVNAA |
| | SEQ ID NO. 63 | SEQ ID NO. 64 | AYPY |
| | | | SEQ ID NO. 65 |
| 7784H1 | GYTSNRYS | IWTDGGRT | AASSLLLQTINPR |
| | SEQ ID NO. 66 | SEQ ID NO. 67 | AWPY |
| | | | SEQ ID NO. 68 |

### Example 2: Design and Expression of MUC17 Antigens

A nucleotide sequence encoding the amino acid sequence (UniProt: Q685B3) of human MUC 17 protein extracellular region truncation was cloned into a His-tagged pTT5 vector (Youflio, VT2202). A plasmid was prepared according to the plasmid extraction kit method and transiently expressed in Expi 293F cells (Gibco, A14527) to obtain the antigen and the protein for detection used in the present application. The method for preparing the cynomolgus monkey MUC 17 extracellular region truncation protein was the same as the method for preparing the human recombinant protein. The cynomolgus monkey MUC17 sequence was from Uniprot No: AOA2K5WHO9. The specific sequence information of the recombinant protein is shown below:

Human MUC17 ECD4131-his (His-tagged human MUC17
protein extracellular region fusion protein)
(SEQ ID NO. 69):
RTTTCFGDGCQNTASRCKNGGTWDGLKCQCPNLYYGELCEEVVSSID
IGPPETISAQMELTVTVTSVKFTEELKNHSSQEFQEFKQTFTEQMNI
VYSGIPEYVGVNITKLRLGSVVVEHDVLLRTKYTPEYKTVLDNATEV
VKEKITKVTTQQIMINDICSDMMCFNTTGTQVQNITVTQYDPEEDCR
KMAKEYGDYFVVEYRDQKPYCISPCEPGFSVSKNCNLGKCQMSLSGP
QCLCVTTETHWYSGETCNQGTQKSLVYGHHHHHH
Cyno MUC17 ECD3577-mFc (mFc-tagged cynomolgus
monkey MUC17 protein extracellular region
fusion protein) (SEQ ID NO. 70):
PLATIPVSTTSLTSSEGSTISTPSVDISTPVTTSIVGDQCSRCKNGG
FWDGLKCQCLTPYYGESCEEVVNSIDIAPPETVSAQMELTVTVTSVK
FTDELKNHSSQEFREFNKTFTEQMNIVYSGIPEYVGVNITNLRLGSV
VVEHDVLLRTKYTPEYKTALDNATEVVKQKITKVTTEQIMTNDNCSA
LMCFNTTGTQVQNITVTQYDPVEECRQKAEEYEDYFLVEYRDQKPYC
ISPCESGFNASKNCNHGKCQMSQNGARCLCVTTETHWYSGEDCNQGT
QKSEPRGPTIKPCPPCKCPAPNLLGGPSVFIFPPKIKDVLMISLSPI
VTCVVVDVSEDDPDVQISWFVNNVEVHTAQTQTHREDYNSTLRVVSA
LPIQHQDWMSGKEFKCKVNNKDLPAPIERTISKPKGSVRAPQVYVLP
PPEEEMTKKQVTLTCMVTDFMPEDIYVEWTNNGKTELNYKNTEPVLD
SDGSYFMYSKLRVEKKNWVERNSYSCSVVHEGLHNHHTTKSFSRTPG
K

### Example 3: Expression and Purification of Bispecific Antibodies

### 3.1 Transfection of BsAb Plasmids

Nucleotide sequences encoding light and heavy chains of AMG199, Bis57, Bis58, Bis59, and Bis92 were each cloned into a pTT5 vector. The plasmid and the transfection reagent PEI (Polysciences, Cat. No. 24765-1) were added to OPTI-MEM (Gibco, Cat. No. 11058021). The mixture was well mixed and left to stand for 15 min. Expi293 cells (manufacturer: Thermofisher, Cat. No. A14527) were added, and the system was incubated on a shaker at 37° C. in 5% CO₂ at 120 rpm. On day 2 of the transfection, OPM-293 ProFeed (Shanghai OPM Biosciences Co., Ltd., Cat. No. F081918-001) and 6 g/L glucose (manufacturer: Sigma, Cat. No. G7528) were added. On day 6 of the transfection, the cell supernatant was collected.

### 3.2 Expression and Purification of Bispecific Antibodies

### 3.2.1 Purification Method for AMG199 Control BsAb

After the culture supernatant was collected, Protein A affinity and molecular sieve purification were performed on the protein using AKTA Pure. The resulting antibody was quantitatively and qualitatively analyzed by SDS-PAGE, SEC-HPLC, and CE-SDS. The specific purification method is as follows: Primary purification was performed using a Protein A column (Mabselect SuRe™, purchased from Cytiva). The Protein A column was first equilibrated with 3-5 column volumes of an equilibration buffer (PBS buffer, pH 7.4), and then the clarified culture supernatant was loaded at a flow rate of 8 mL/min. After the loading was completed, the column was rinsed with 3-5 column volumes of a high-salt rinsing solution (20 mM phosphate-buffered saline, 1 M NaCl, pH 7.4). The protein bound to the Protein A column was eluted with an eluent (20 mM citrate buffer, pH 3.5), and the elution of the protein was monitored according to an A280 ultraviolet absorption peak. The eluted protein was collected, neutralized to pH 5-6 by adding 1 M Tris-HCl at pH 8.0, and dialyzed against a molecular sieve buffer (10 mM Hac, 150 mM NaCl, pH 5.5). In the next step, the sample was subjected to polishing purification with a molecular sieve (purchased from Bestchrom), and the target sample was collected, concentrated, and exchanged into a 559 buffer (10 mM Hac, 9% sucrose, pH 5.5) by dialysis.

The system was subjected to sterile filtration using a 0.22 m filter and aseptically stored, thus obtaining the purified AMG199 antibody.

### 3.2.2 Purification Method for Bispecific Antibodies

After the culture supernatant was collected, Protein A affinity and KappaSelect affinity purification were performed on the protein using AKTA Pure. The resulting antibodies were each quantitatively and qualitatively analyzed by SEC-HPLC and CE-SDS. The specific purification method is as follows.

1. Primary purification was performed using a Protein A column (Mabselect SuRe™, purchased from Cytiva). The Protein A column was equilibrated with 3-5 column volumes of an equilibration buffer (PBS buffer, pH 7.4), and then the clarified culture supernatant was loaded at a flow rate of 8 mL/min. After the loading was completed, the column was rinsed with 3-5 column volumes of a high-salt rinsing solution (20 mM phosphate-buffered saline, 1 M NaCl, pH 7.4). The protein bound to the Protein A column was eluted with an eluent (20 mM citrate buffer, pH 3.5), and the elution of the protein was monitored according to an A280 ultraviolet absorption peak. The eluted protein was collected and neutralized to pH 5-6 by adding 1 M Tris-HCl at pH 8.0.
2. Polishing purification was performed using HiTrap KappaSelect (purchased from Cytiva). The column was first equilibrated with 3-5 column volumes of an equilibration buffer (PBS buffer, pH 7.4), and then the primary pure protein solution was loaded at a flow rate of 5 mL/min. After the loading was completed, the column was rinsed with 3-5 column volumes of PBS and then 10 column volumes of 10 mM phosphate-buffered saline (pH 7.4). The protein bound to the KappaSelect column was eluted with an eluent (50 mM glycine, pH 3.4), and the elution of the protein was monitored according to an A280 ultraviolet absorption peak. The eluted protein was collected and neutralized to pH 5-6 by adding 1 M Tris-HCl at pH 8.0. After concentration, the sample was exchanged into a 559 buffer (10 mM Hac, 9% sucrose, pH 5.5) by dialysis. The system was subjected to sterile filtration using a 0.22 m filter and aseptically stored, thus obtaining the purified bispecific antibody.

### 3.3 Purity Detection of Bispecific Antibodies

### 3.3.1 SEC-HPLC Analysis

The test samples were analyzed using an SEC-HPLC method to characterize the molecular size uniformity of the bispecific antibodies and determine the purities of the bispecific antibodies. The HPLC used in this method was Agilent 1260, the chromatographic column was TSKgel G3000SWXL (purchased from Tosoh Bioscience), the mobile phase was 200 mM phosphate-buffered saline, pH 7.0/isopropanol (v/v 9:1) (Batch No. 20220616101), the detection temperature was 25° C., the flow rate was 0.5 mL/min, the detection wavelength was 280 nm, the target protein was subjected to 10-fold dilution with DI water, the loading amount was 50 g, and the analysis time was 40 min. For the SEC-HPLC data, the chromatogram was analyzed using a manual integration method, and the protein purity was calculated according to an area normalization method. The main peak was considered as a monomer, the chromatographic peak preceding the main peak was referred to as an aggregate, and the chromatographic peak following the main peak was referred to as a fragment. The purity information of the obtained diabodies is shown in Table 4 below.

TABLE 4
Purity of bispecific antibodies as detected by SEC-HPLC

| Antibody name | Monomer (%) | Aggregate (%) | Fragment (%) |
|---|---|---|---|
| Bis 57 | 97.87 | 2.13 | / |
| Bis 58 | 98.35 | 1.53 | 0.12 |
| Bis 59 | 99.35 | 0.65 | / |
| Bis 92 | 98.26 | 1.49 | 0.26 |
| AMG199 | 99.63 | 0.37 | / |

### 3.3.2 CE-SDS Analysis

The test samples were analyzed using a non-reduced CE-SDS method to determine the purities of the bispecific antibodies and characterize the size uniformity of the test samples. The capillary electrophoresis apparatus used in this method was AB Sciex PA 800 Plus, the detector was PDA, the detection wavelength was 220 nm, the effective length for capillary detection was 20 cm, the protein separation voltage was 15.0 kV, and the NR-CE SDS detection time was 40 min. The amount of the test sample was 100 g. For the NR-CE-SDS method, an SDS sample buffer (20 mM PB, 5 mM citric acid, 1% SDS, pH 6.5) was added to the protein sample and dissolved to 94 μL. 5 μL of 100 mmol/L NEM and 1 μL of 10 KD marker were added, and the mixture was incubated at 70° C. for 10 min to obtain the test sample for NR-CE-SDS. For the NR-CE SDS data, the chromatogram was analyzed using a manual integration method, and the protein purity was calculated according to an area normalization method. The purity information of the obtained bispecific antibodies is shown in Table 5 below.

TABLE 5
Purity of bispecific antibodies as detected by NR-CE-SDS

| Antibody name | Main Peak (%) | Fragment (%) | Aggregate (%) |
|---|---|---|---|
| Bis 57 | 98.45 | 1.55 | / |
| Bis 58 | 98.17 | 1.83 | / |
| Bis 59 | 98.49 | 1.51 | / |
| Bis 92 | 99.02 | 0.98 | / |
| AMG199 | 99.63 | 0.37 | / |

### Example 4: Assay for Binding Activity of Bispecific Antibodies

### 4.1 Binding of Bispecific Antibodies to Human and Monkey MUC17 Proteins as Detected by ELISA

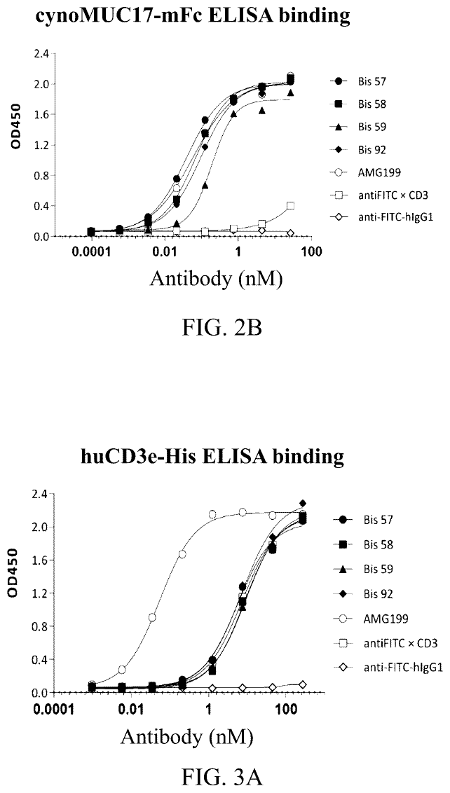

A human MUC17-his protein was diluted with PBS to a final concentration of 2 g/mL and then added to a 96-well ELISA plate at 50 μL/well. The plate was incubated at 4° C. overnight. The next day, the plate was washed twice with PBST, and a blocking buffer [PBS+2% (w/w) BSA] was added for blocking at room temperature for 2 h. The blocking buffer was discarded, and the bispecific antibodies and the positive and negative control antibodies (diluted in a 6-fold gradient from a starting concentration of 27 nM) were each added at 50 μL/well. After incubation at 37° C. for 1 h, the plate was washed 3 times with PBST. A horseradish peroxidase (HRP)-labeled secondary antibody (purchased from Merck, Cat. No. AP113P) was added for incubation at 37° C. for 1 h, and the plate was washed 5 times with PBST. A TMB substrate was added at 50 μL/well for incubation at room temperature for 10 min, and then a stop solution (1.0 M HCl) was added at 50 μL/well. An ELISA plate reader (Multimode Plate Reader, EnSight, purchased from Perkin Elmer) was used to read OD₄₅₀ m values, and the binding activity of the bispecific antibodies to the human MUC17 protein is shown in FIG. 2A. The results show that the negative controls antiFITCxCD3 and anti-FITC-hIgG1 (derived from J Biol Chem. Jan. 5, 1990; 265(1):133-8) did not bind to MUC17, and the bispecific antibodies Bis57, Bis58, Bis59, and Bis92 all effectively bound to the human MUC17-his protein.

The monkey MUC17-mFc protein was subjected to ELISA and data analysis according to the method described in Example 4.1. The analysis results are shown in FIG. 2B. The bispecific antibodies Bis57, Bis58, Bis59, and Bis92 all had relatively good binding activity to the monkey MUC17 protein.

### 4.2 Binding of Bispecific Antibodies to Human and Monkey CD3e Proteins as Detected by ELISA

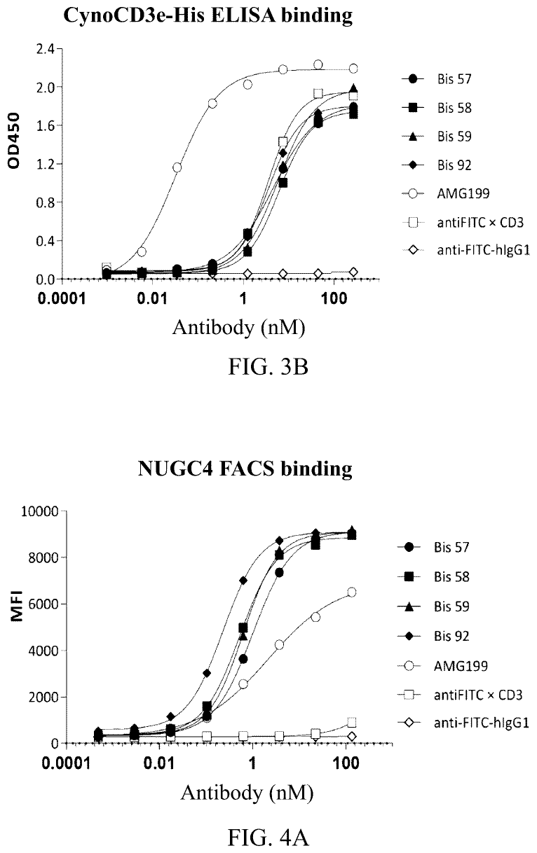

Human CD3e-His protein (Sino Biological, CAT #10977-H08H) was diluted to a final concentration of 1 g/mL in PBS, added to a 96-well ELISA plate at 50 μL/well, and then incubated at 4° C. overnight. The next day, the plate was washed twice with PBST, and a blocking buffer [PBS+2% (w/w) BSA] was added for blocking at room temperature for 2 h. The blocking buffer was discarded, and the bispecific antibodies and the positive and negative control antibodies (diluted in a 6-fold gradient from a starting concentration of 270 nM) were each added at 50 μL/well. After incubation at 37° C. for 1 h, the plate was washed 3 times with PBST. A horseradish peroxidase (HRP)-labeled secondary antibody (purchased from Merck, Cat. No. AP113P) was added for incubation at 37° C. for 1 h, and the plate was washed 5 times with PBST. A TMB substrate was added at 50 μL/well for incubation at room temperature for 10 min, and then a stop solution (1.0 M HCl) was added at 50 μL/well. An ELISA plate reader (Multimode Plate Reader, EnSight, purchased from Perkin Elmer) was used to read OD_{450 nm} values, and the binding activity of the bispecific antibodies to the human CD3e protein is shown in FIG. 3A. The results show that the negative control anti-FITC-hIgG1 did not bind to CD3e, and the bispecific antibodies Bis57, Bis58, Bis59, and Bis92 all effectively bound to the human CD3e-his protein.

The monkey CD3-his protein (ACRO, CAT #CDE-C5226) was subjected to ELISA and data analysis according to the method described in Example 4.2. The analysis results are shown in FIG. 3B. The bispecific antibodies Bis57, Bis58, Bis59, and Bis92 all had relatively good binding activity to the monkey CD3e protein.

### 4.3 Binding of Bispecific Antibodies to Human MUC17 as Detected by Flow Cytometry Assay (FACS)

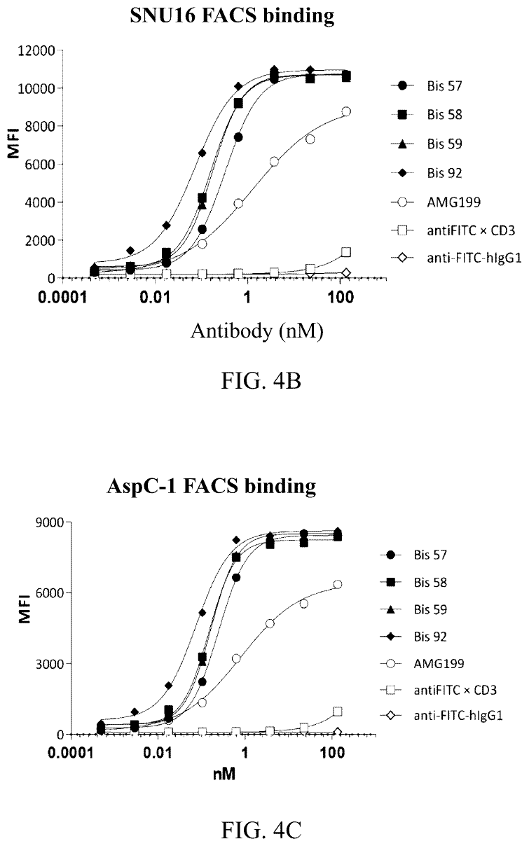

The endogenous tumor cells NUGC4 were expanded to the logarithmic growth phase in a T-175 culture flask, the medium was removed by pipetting, and the cells were washed twice with a PBS buffer and digested with trypsin. Then, a complete medium was added to stop the digestion, and the cells were blown into a single cell suspension. After cell counting, the cells were centrifuged, and the cell pellet was resuspended to 2×10⁶ cells/mL in an FACS buffer (PBS+2% fetal bovine serum), and added to a 96-well FACS reaction plate at 100 μL/well. The plate was centrifuged, and the supernatant was discarded. The test antibody samples (diluted in a 3-fold gradient from a starting concentration of 100 nM) were each added at 50 μL/well and uniformly mixed with the cells, and the mixture was incubated at 4° C. for 1 h. After the plate was centrifuged and washed 3 times with a PBS buffer, 50 μL of Alexa Fluor® 647 AffiniPure Goat Anti-Human IgG, Fc7 fragment specific-labeled secondary antibody (purchased from Jackson, Cat. No. 109-605-098) was added to each well, and the mixture was incubated at 4° C. for 1 h. The results were detected and analyzed by FACS (FACS Canto™, purchased from BD) after the plate was centrifuged and washed 3 times with a PBS buffer and the cells were resuspended in 100 μL of PBS. Data analysis was performed by software (FlowJo) to obtain the mean fluorescence intensity (MFI) of the cells. Then, analysis was performed by software (GraphPad Prism8), data were fitted, and EC50 values were calculated. As shown in FIG. 4A, the bispecific antibodies Bis57, Bis58, Bis59, and Bis92 were all able to specifically bind to NUGC4 cells.

The same method was used to detect the binding of the bispecific antibodies to the gastric cancer cell line SNU16 and the pancreatic cancer cell line ASPC1 (FIG. 10, showing the expression levels of MUC17 in NUGC4 cells, SNU16 cells, and ASPC1 cells), with moderate expression of MUC17. The results show that the bispecific antibodies Bis57, Bis58, Bis59, and Bis92 were all able to have good binding activity to SNU16 (FIG. 4B) and ASPC-1 (FIG. 4C), the binding abilities of which were stronger than that of AMG199.

### 4.4 Binding of Bispecific Antibodies to Cells Overexpressing MUC17 as Detected by Flow Cytometry Assay (FACS)

A nucleotide sequence encoding the human MUC17 fragment was cloned into a pcDNA5 vector (purchased from General). A plasmid was prepared, and an overexpression cell line was constructed. A monoclonal cell line with a relatively high fluorescence intensity was selected for subsequent detection. The constructed overexpression cell line was designated as FlpinCHO-huMUC17-D1.

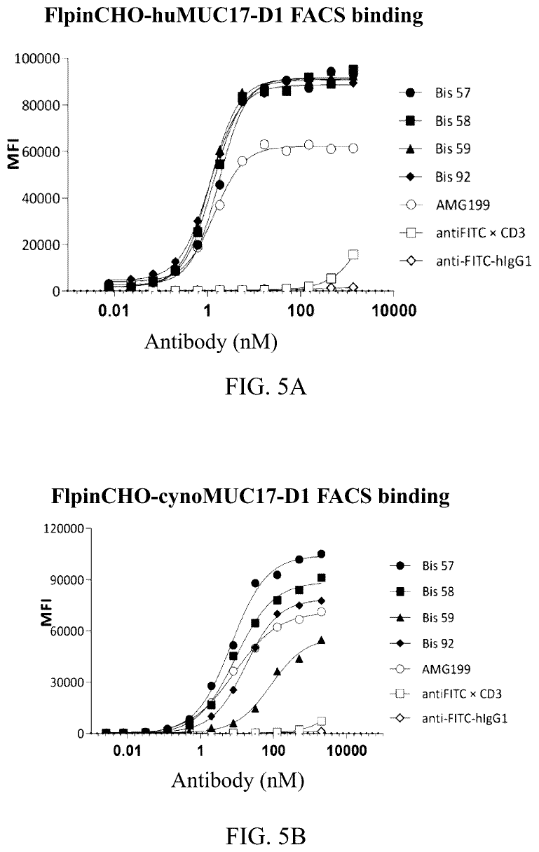

FlpinCHO-huMUC17-D1 was subjected to FACS assay and data analysis according to the method described in Example 4.3. The test antibody samples were diluted in a 3-fold gradient from a starting concentration of 1350 nM. The results are shown in FIG. 5A. The bispecific antibodies Bis57, Bis58, Bis59, and Bis92 were able to effectively bind to FlpinCHO-huMUC17-D1 cells. Recombinant cells overexpressing monkey MUC17 (FlpinCHO-cynoMUC17-D1) were subjected to FACS assay and data analysis according to the same method. The results are shown in FIG. 5B. The bispecific antibodies Bis57, Bis58, Bis59, and Bis92 were able to effectively bind to FlpinCHO-cynoMUC17-D1 cells.

### 4.5 Binding of Bispecific Antibodies to Jurkat Cells as Detected by Flow Cytometry Assay (FACS)

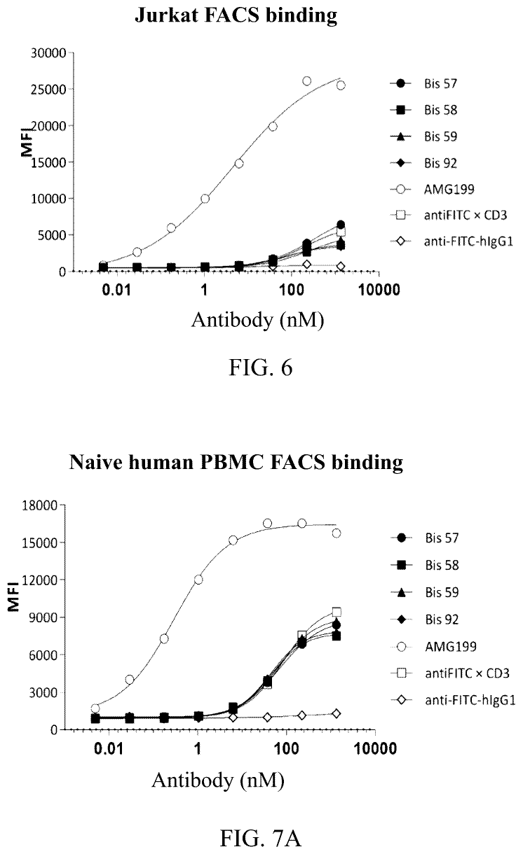

To detect the binding ability of the bispecific antibodies to the cell surface CD3, binding assays on Jurkat cells were performed by FACS. Jurkat cells were cultured and collected. The test antibody samples were diluted in a 3-fold gradient from a starting concentration of 1350 nM and subjected to FACS assay and data analysis according to the method described in Example 4.3. The assay results are shown in FIG. 6. The bispecific antibodies Bis57, Bis58, Bis59, and Bis92 were able to effectively bind to Jurkat cells, the binding abilities of which were weaker than that of AMG199. The weak binding to CD3 may help to alleviate the side effects of the drugs.

### 4.6 Binding of Bispecific Antibodies to Human and Monkey PBMCs as Detected by Flow Cytometry Assay (FACS)

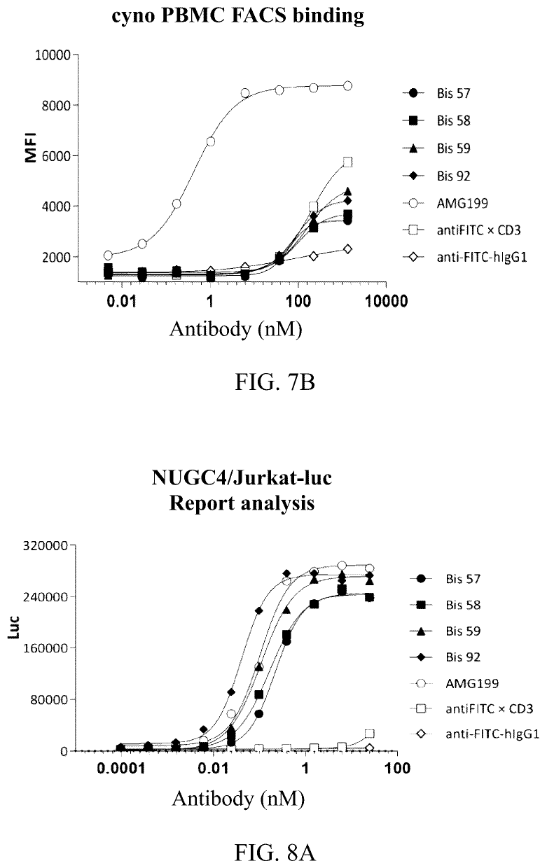

After overnight culture, human PBMCs (SailyBio, CAT #XFB-HP010B) and monkey PBMCs (Hkeybio, CAT #5208) were collected, Fc Block (BD, 564220) was added, and the samples were subjected to FACS assay and data analysis according to the method in Example 4.3. The results are shown in FIGS. 7A to 7B. The bispecific antibodies Bis57, Bis58, Bis59, and Bis92 were able to effectively bind to human and monkey PBMCs, the binding abilities of which were weaker than that of AMG199. The weaker binding ability to CD3 helps to alleviate the cytokine release syndrome induced by drugs.

### 4.7 Effect of Bispecific Antibodies on T Cell Activity as Detected by Luciferase Reporter Gene Assay

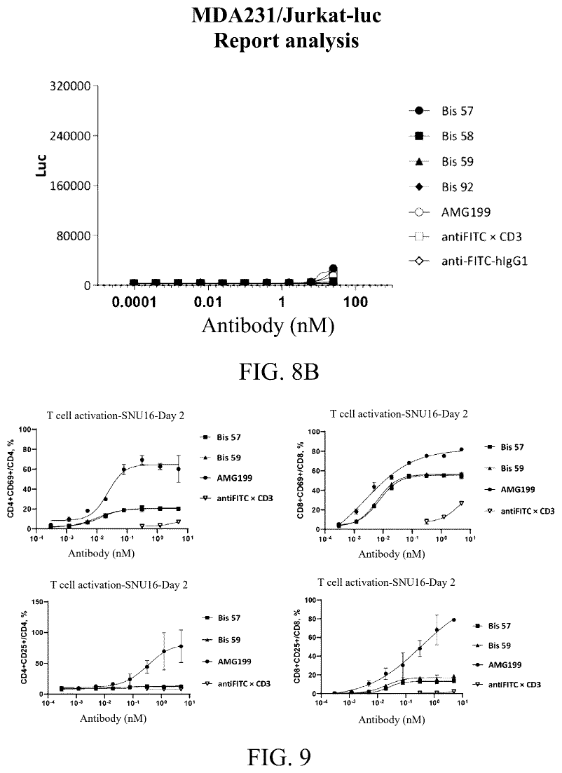

NUGC4 (MUC17-positive cells) and Jurkat-luc cells (overexpressing the luciferase gene on the basis of Jurkat cells) were cultured and collected, and separately resuspended to 5×10⁴ cells/25 μL. The test antibody samples were diluted in a 4-fold gradient from a starting concentration of 25 nM. Meanwhile, 25 μL of NUGC4 cells and 25 μL of Jurkat-luc cells were mixed with 50 μL of antibody dilutions and added to a 96-well white clear-bottom microplate (coming, CAT #3610). The plate was incubated at 37° C. for 5 h, and then 50 μL of Nano-Glo Luciferase reagent (Promega, CAT #N1130) was added. The plate was shaken and incubated at room temperature at 450 rpm for 10 min, and then the results were read using an Envision microplate reader (Perkin Elmer, Envision2105). The results are shown in FIG. 8A. The bispecific antibodies Bis57, Bis58, Bis59, and Bis92 were all able to effectively activate Jurkat cells. The same method was used to detect the activation after MDA-231 (MUC17-negative cells) and Jurkat-luc co-incubation. The results are shown in FIG. 8B, indicating that none of the antibodies can activate Jurkat cells. This suggests that the activation of T cells by the MUC17/CD3 bispecific antibodies is specifically mediated by MUC17.

### 4.8 Activation of T Cells by Bispecific Antibodies in Tumor Cell Killing Assay In Vitro

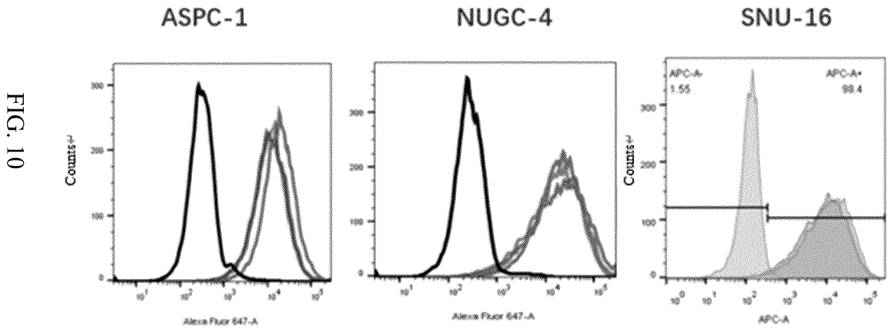

The cells were seeded in a 96-well plate in a ratio of the tumor cells SNU16 to PBMCs (purchased from Allcells, Cat. No. PBO04F-C) of 1:10, with the tumor cells at 5000 cells/50 μL/well and the PBMCs at 50,000 cells/50 μL/well, and the total volume was 100 μL/well. The test antibodies were each diluted to the desired concentration in an RPMI-1640 medium and added to the cell plate at 10 μL/well. The plate was incubated in a 5% CO₂ incubator at 37° C. for 48 h. After 48 h, the cell culture plate was taken out and left to stand until it returned to room temperature. The cell plate was then centrifuged at 350 g for 7 min, and the supernatant was discarded. The plate was rinsed once with 100 μL of PBS and centrifuged at 350 g for 5 min. After the centrifugation was completed, the supernatant was discarded, and this step was repeated once. The test antibodies were each diluted with PBS containing 1% BSA in a ratio of 1:60, and 60 μL of the above test antibody dilution was added to each well. The cells were stained at 4° C. for 30 min, wherein the antibody FITC anti-human CD3 (purchased from BD, Cat. No. 300440) was used to label T cells, the antibody APC anti-human CD4 (purchased from BD, Cat. No. 300537) was used to label CD4-positive cells, the antibody Brilliant Violet 421™ anti-human CD69 (purchased from BD, Cat. No. 310930) was used to label CD69-positive cells, and the antibody PE anti-human CD25 (purchased from BD, Cat. No. 302606) was used to label CD25-positive cells. After the staining was completed, the plate was centrifuged at 350 g for 5 min, and the supernatant was discarded. The cells were rinsed twice with PBS. Then, flow cytometry was directly performed. As shown in FIG. 9, in the T cell activation experiment, the up-regulation effects of both Bis 57 and Bis 59 on CD69 and CD25 on the surface of CD4-positive T cells and CD8-positive T cells were weaker than that of the control antibody AMG199.

### Example 5: Affinity Assay on Bispecific Antibodies

The strength of antibody-antigen binding was assayed with a BIAcore 8K instrument using a Protein A capture method. First, Protein A was immobilized onto a CM4 chip (purchased from GE, BR-1005-34) using an amino coupling method. According to the instruction of the Amine Coupling Kit (purchased from GE, BR100633), NHS and EDC were mixed, with HBS-EP+ pH 7.4 as a mobile phase, to activate the chip for about 600 s; the Protein A was diluted to 50 g/mL with 10 mM sodium acetate (pH 4.5) and injected for 600 s, and finally, the remaining activated sites were blocked with ethanolamine. Then, the affinity of the antibody for the antigen was assayed using a multi-cycle kinetic method. In each cycle, first, the test antibody was captured using the Protein A chip, and then a single concentration of the antigen protein was injected. The association and dissociation processes of the antibody with the antigen protein were recorded, and finally, the chip was regenerated using Glycine (pH 1.5), wherein the mobile phase was HBS-EP+ (10 mM HEPES, 150 mM NaCl, 3 mM EDTA, 0.05% surfactant P20), the flow rate was 30 μL/min, the regeneration time was 30 s, and the assay temperature was 25° C. Finally, according to a 1:1 binding model, the data were analyzed, and the antibody-antigen binding kinetics parameters, including the association rate constant Ka, the dissociation rate constant Kd, the equilibrium dissociation constant KD, and the maximum binding signal Rmax, were fitted. The association rates (Ka), dissociation rates (Kd), and binding affinities (KD) of the bispecific antibodies Bis 57, Bis 58, Bis 59, Bis 92, and AMG199 to the MUC17 proteins are shown in Table 6.

TABLE 6
Affinity of bispecific antibodies for MUC17 proteins as detected by SPR (Biacore)

| Antibody | Human MUC17-ECD(4131-4393)-His protein | | | Monkey MUC17-ECD3577-mFc protein | | |
|---|---|---|---|---|---|---|
| name | ka (1/Ms) | kd (1/s) | KD (M) | ka (1/Ms) | kd (1/s) | KD (M) |
| Bis 57 | 1.68E+05 | 4.86E−05 | 2.89E−10 | 3.39E+04 | 1.60E−04 | 4.71E−09 |
| Bis 58 | 3.64E+05 | 1.38E−04 | 3.78E−10 | 4.36E+04 | 3.10E−04 | 7.11E−09 |
| Bis 59 | 4.45E+05 | 9.74E−05 | 2.19E−10 | 3.39E+04 | 8.66E−04 | 2.56E−08 |
| Bis 92 | 5.72E+05 | 7.81E−05 | 1.36E−10 | 3.81E+04 | 5.11E−04 | 1.34E−08 |
| AMG199 | 2.17E+05 | 1.75E−03 | 8.06E−09 | 6.07E+04 | 5.49E−04 | 9.05E−09 |

### Example 6: Bispecific Antibody-Mediated In Vitro Tumor Cell Killing

The killing experiment of bispecific antibody-mediated PBMCs on tumor cells was achieved by quantitatively detecting the cell proliferation. Cell Titer glo was used to detect the content of ATP in cells, where ATP is an indicator of the metabolism of living cells and is directly proportional to the number of cells in the culture. Four different tumor cells were used, including three tumor cell lines with moderate expression of MUC17 (SNU16, NUGC4, and ASPC-1, and the expression levels of MUC17 are shown in FIG. 10) and one MUC17-negative control cell line MDA-MB-231.

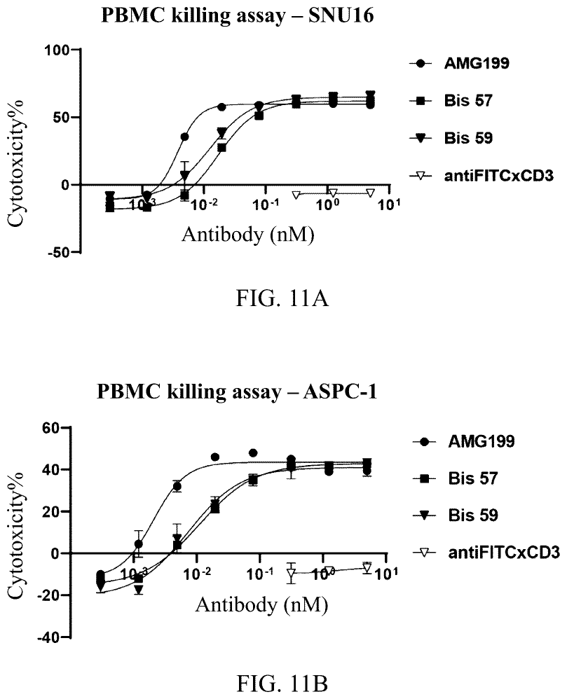

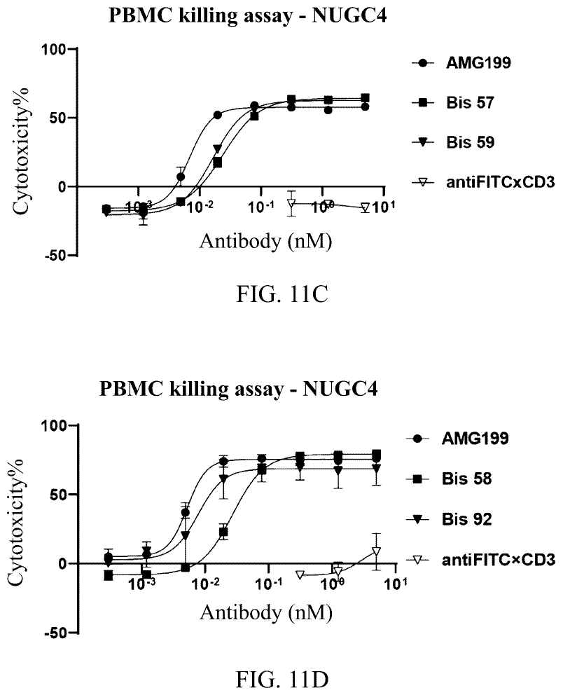

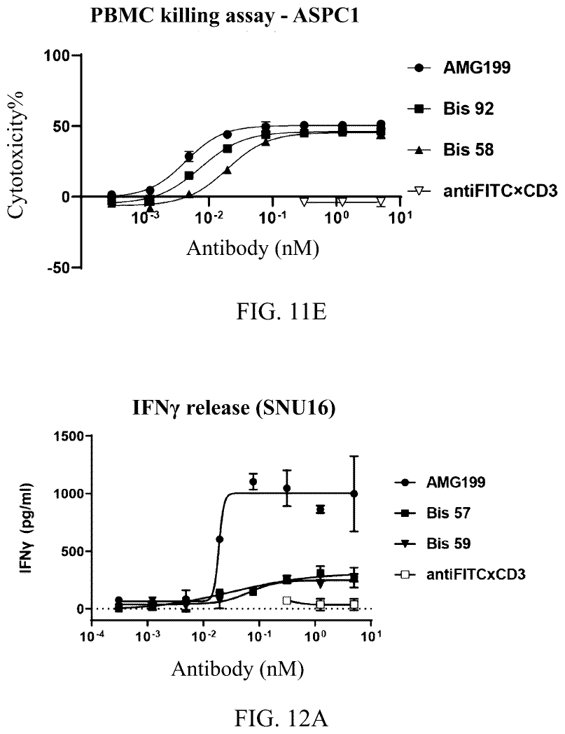

Tumor cells at 5000 cells/well and PBMCs (Allcells, PB004F-C) at 50,000 cells/well were diluted with an RPMI Medium 1640 medium containing 10% FBS and added to a 96-well plate. The control antibody and the test antibodies were diluted to different concentrations in the RPMI Medium 1640 medium and added to the 96-well plate. The antibodies in the reaction system were diluted in a 4-fold gradient starting from 5 nM to their final concentrations. After the CD3 BsAb molecules were incubated with PBMCs and tumor cells in a 5% CO₂ incubator at 37° C. for 2 days, ATP in the living cells was quantified using a CTG kit (Promega, G7573) to reflect the antibody-mediated killing of the tumor cells by the PBMCs. Killing rate of tumor cells=(100×(medium well−experimental well)/(medium well−PBMC well))%. The killing results are shown in FIGS. 11A to 11E. The bispecific antibodies Bis57, Bis58, Bis59, and Bis92 all had significant killing effects on MUC17-positive tumor cells. The specific killing data are shown in Table 7 below.

TABLE 7
PBMC-mediated in vitro tumor cell killing EC50

| | Cell line | | |
|---|---|---|---|
| Antibody | SNU16 | ASPC-1 | NUGC4 |
| AMG199 | 0.003789 nM | 0.002067 nM | 0.006819 nM |
| Bis57 | 0.01702 nM | 0.01097 nM | 0.02398 nM |
| Bis58 | NT | 0.01937 nM | 0.02713 nM |
| Bis59 | 0.01260 nM | 0.007296 nM | 0.01659 nM |
| Bis92 | NT | 0.007394 nM | 0.007792 nM |

Note:
NT represents not evaluated

### Example 7: Bispecific Antibody-Mediated Cytokine Secretion

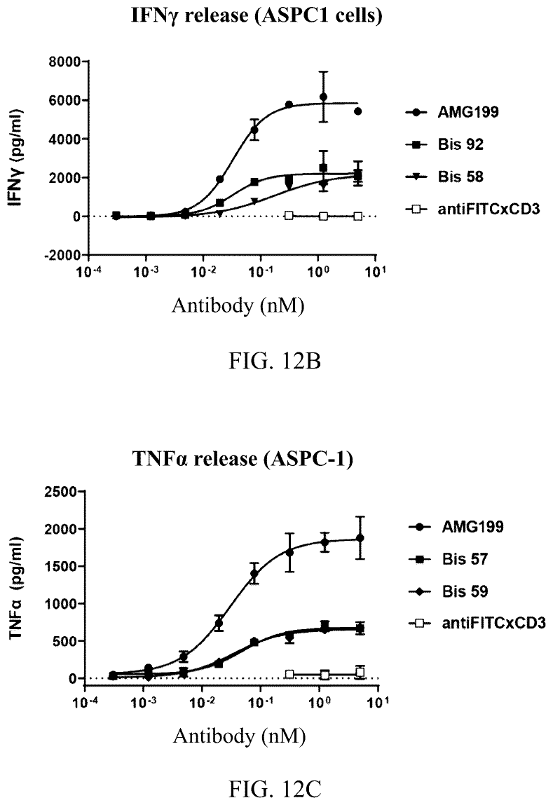

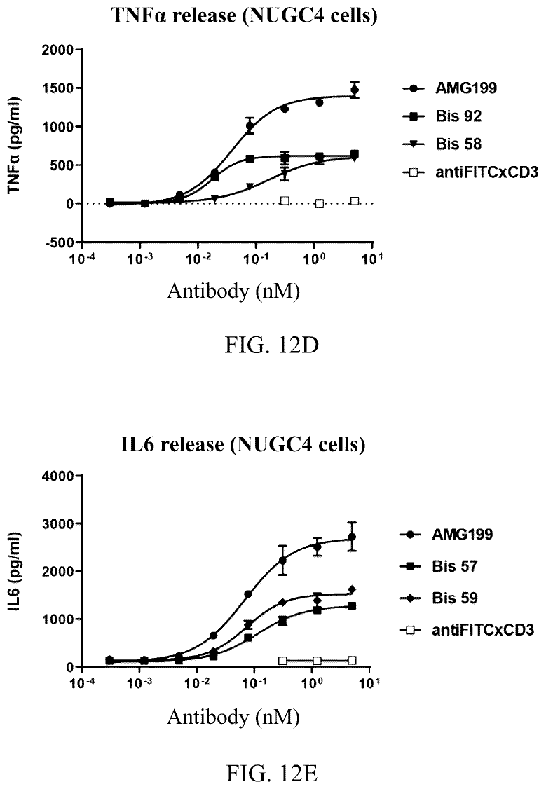

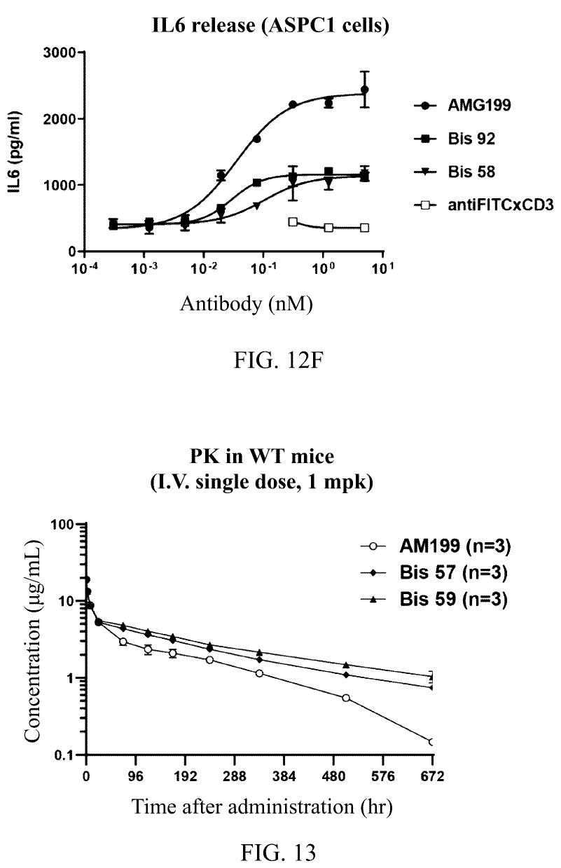

T cells are activated under the mediation of bispecific antibodies to release cytokines while killing target cells. The cell supernatants in Example 6 were collected, centrifuged at 3000 rpm for 10 min, and cryopreserved in a refrigerator at −80° C. for later use. The secretion levels of cytokines IFNγ (Cisbio, 62HIFNGPEH) and TNFα (Cisbio, 62HTNFAPEH) in the cell supernatants were determined by HTRF. The secretion level of IL-6 (BD, 555220) was measured by ELISA. The procedures are detailed in the instruction of the kit. The results are shown in FIGS. 12A to 12F. The bispecific antibodies Bis57, Bis58, Bis59, and Bis92 were all able to effectively induce secretion of IFNγ, TNFα, and IL6 from PBMCs when the PBMCs and MUC17-positive tumor cells coexisted, and the cytokine secretion amounts were significantly lower than that of AMG199. The release level of the cytokine reflects the activity of the multispecific antibody, and is generally positively correlated with the activation level of T cells, in vitro killing activity, and in vivo anti-tumor activity. Meanwhile, the stronger the T cell activation activity of the multispecific antibody, the higher the risk of producing a cytokine release syndrome (CRS) after entering the human body.

### Example 8: Pharmacokinetics of Bispecific Antibodies in Mice

To compare the pharmacokinetic differences among different antibodies, in this example, SPF-grade female wild-type C57 mice aged 6-8 weeks and weighing about 18-20 g were used, with 3 mice in each group. The control antibody (AMG199) and the bispecific antibodies (Bis57 and Bis59) prepared in Example 3 were administered by single injection via the tail vein at a dose of 1 mg/kg.

The mice were fed with standard feed and had free access to food and water. The drugs were diluted with physiological saline. Blood was collected from the orbit at time points of before administration and 0.25 h, 2 h, 8 h, 24 h, 72 h, 120 h, 168 h, 240 h, 336 h, 504 h, and 672 h after administration. Blood samples were collected in micro blood collection tubes, and the tubes were left to stand for about 30 min and then centrifuged at 12,000 rpm at 4° C. for 5 min. Serum was separated into low-adhesion centrifuge tubes with compound codes and time points indicated, and cryopreserved at −80° C. before analysis.

The sample was coated with the human MUC17 protein, and the concentrations of the antibodies in the serum of the control group and the test groups were determined by an indirect enzyme-linked immunosorbent assay. Pharmacokinetic parameters were calculated based on the plasma concentration of each animal at different time points. The specific results are shown in Table 8 and FIG. 13. The results show that the molecules of the present application have good PK performance. The half-lives of Bis 57 and Bis59 were 248 h and 309 h, respectively, which were significantly higher than that of the control group AMG199 of 130 h. The terminal exposures of Bis 57 and Bis59 were 1575 h*μg/mL and 1825 h*μg/mL, respectively, which were slightly higher than that of AMG199 of 1118 h*μg/mL.

TABLE 8
Pharmacokinetic parameters in wild-type C57 mice

| | Half-life, | Tmax | Cmax | AUClast |
|---|---|---|---|---|
| Drug | hour | (h) | (μg/mL) | (h*μg/mL) |
| Bis57 | 248. | 0.25 | 18.90 | 1575 |
| Bis59 | 309. | 0.25 | 20.33 | 1825 |
| AMG199 | 130. | 0.25 | 18.97 | 1118 |

### Example 9: Pharmacodynamic Model of PBMC-Reconstituted Mice

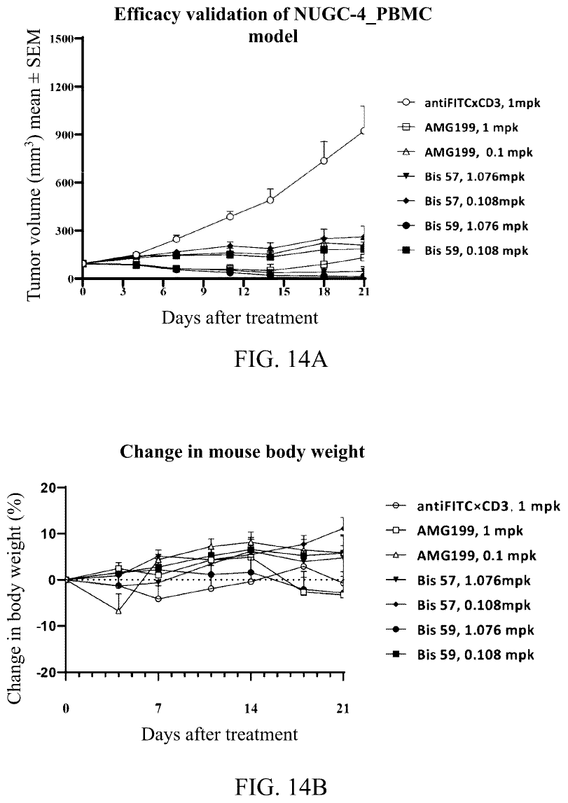

An NPG mouse (NPG: female, aged 5-6 weeks, Beijing Vitalstar Biotechnology Co., Ltd.) model reconstituted with human PBMCs was used to evaluate the anti-tumor efficacy of the bispecific antibodies in vivo. NUGC4 cells were cultured to the logarithmic growth phase and centrifuged, and the cells were collected and subcutaneously inoculated into the mice at 10×10⁶ cells/mouse. On the same day, human PBMC cells were thawed and intravenously inoculated into the mice at 5×10⁶ cells/mouse. The mice were then fed normally. When the tumor volume of the tumor-bearing mice reached about 100 mm³, the mice were randomly grouped with 8 mice in each group, and the reconstitution rate was detected at the same time. The day of grouping was defined as day 0 of the experiment. The test antibodies and the control antibody were then administered intravenously twice a week at an equimolar dose, 6 times in total. The tumors were observed and measured twice a week, and the tumor volume was calculated according to the following formula: tumor volume (TV)=½ (height×width²), where the width is the smaller of the two measurements and the height is defined as the larger of the measurements. During the entire administration period, the body weight of the mice was recorded twice a week, and the changes in the body weight of the mice were calculated. The results are shown in FIGS. 14A and 14B. One week after administration, the bispecific antibodies Bis57 and Bis59 were both able to well inhibit the tumor growth, and there was no significant change in the body weight of the mice during the administration period.

TABLE 9
Effects of test substances on tumor volume of human
PBMC mice implanted with NUGC4 cells

| | | | Volume before | Tumor volume on | Tumor growth |
|---|---|---|---|---|---|
| | Dose, | Administration | administration | day 21 | inhibition |
| Group | mpk | regimen | mm³ | mm³ | TGI % |
| antiFITCxCD3 | 1 | IV, BIW*3 | 93.2 ± 4.3 | 922.8 ± 154.9 | — |
| AMG199 | 1 | IV, BIW*3 | 93.5 ± 4.7 | 131.2 ± 117.9 | 95.46% |
| AMG199 | 0.1 | IV, BIW*3 | 93.2 ± 4.9 | 209.6 ± 33.4 | 85.96% |
| Bis 57 | 1.076 | IV, BIW*3 | 94.5 ± 4.9 | 46.1 ± 29.3 | 105.83% |
| Bis 57 | 0.108 | IV, BIW*3 | 94.2 ± 5.2 | 261.3 ± 67.1 | 79.85% |
| Bis 59 | 1.076 | IV, BIW*3 | 93.6 ± 5.9 | 14.1 ± 2.0 | 109.58% |
| Bis 59 | 0.108 | IV, BIW*3 | 94.5 ± 5.4 | 184.8 ± 42.5 | 89.11% |

1. An anti-MUC17/anti-CD3 bispecific antibody, comprising:
(a) a first antigen-binding moiety comprising a heavy chain variable region (VH) and a light chain variable region (VL), wherein the VH and VL form an anti-CD3 antigen-binding domain, wherein the anti-CD3 antigen-binding domain comprises an HCDR1, an HCDR2, and an HCDR3 in a VH set forth in SEQ ID NO. 9, and an LCDR1, an LCDR2, and an LCDR3 in a VL set forth in SEQ ID NO. 10; and
(b) a second antigen-binding moiety comprising a VHH specifically binding to MUC17, wherein the VHH comprises a CDR1, a CDR2, and a CDR3 from the sequence set forth in SEQ ID NO. 11, or a CDR1, a CDR2, and a CDR3 from the sequence set forth in SEQ ID NO. 12, or a CDR1, a CDR2, and a CDR3 from the sequence set forth in SEQ ID NO. 13, or a CDR1, a CDR2, and a CDR3 from the sequence set forth in SEQ ID NO. 14.

2. The bispecific antibody according to claim 1, wherein:
(1) based on the Kabat numbering scheme, in the first antigen-binding moiety, the HCDR1 comprises the sequence set forth in SEQ ID NO. 15; the HCDR2 comprises the sequence set forth in SEQ ID NO. 16; and the HCDR3 comprises the sequence set forth in SEQ ID NO. 17; or
based on the Chothia numbering scheme, in the first antigen-binding moiety, the HCDR1 comprises the sequence set forth in SEQ ID NO. 33; the HCDR2 comprises the sequence set forth in SEQ ID NO. 34; and the HCDR3 comprises the sequence set forth in SEQ ID NO. 35; or
based on the IMGT numbering scheme, in the first antigen-binding moiety, the HCDR1 comprises the sequence set forth in SEQ ID NO. 51; the HCDR2 comprises the sequence set forth in SEQ ID NO. 52; and the HCDR3 comprises the sequence set forth in SEQ ID NO. 53; and/or
(2) based on the Kabat numbering scheme, in the first antigen-binding moiety, the LCDR1 comprises the sequence set forth in SEQ ID NO. 18; the LCDR2 comprises the sequence set forth in SEQ ID NO. 19; and the LCDR3 comprises the sequence set forth in SEQ ID NO. 20; or
based on the Chothia numbering scheme, in the first antigen-binding moiety, the LCDR1 comprises the sequence set forth in SEQ ID NO. 36; the LCDR2 comprises the sequence set forth in SEQ ID NO. 37; and the LCDR3 comprises the sequence set forth in SEQ ID NO. 38; or
based on the IMGT numbering scheme, in the first antigen-binding moiety, the LCDR1 comprises the sequence set forth in SEQ ID NO. 54; the LCDR2 comprises the sequence set forth in SEQ ID NO. 55; and the LCDR3 comprises the sequence set forth in SEQ ID NO. 56.

3. The bispecific antibody according to claim 1, wherein:
based on the Kabat numbering scheme, in the second antigen-binding moiety, the CDR1 comprises the sequence set forth in SEQ ID NO. 21; the CDR2 comprises the sequence set forth in SEQ ID NO. 22; and the CDR3 comprises the sequence set forth in SEQ ID NO. 23; or
based on the Kabat numbering scheme, in the second antigen-binding moiety, the CDR1 comprises the sequence set forth in SEQ ID NO. 24; the CDR2 comprises the sequence set forth in SEQ ID NO. 25; and the CDR3 comprises the sequence set forth in SEQ ID NO. 26; or
based on the Kabat numbering scheme, in the second antigen-binding moiety, the CDR1 comprises the sequence set forth in SEQ ID NO. 27; the CDR2 comprises the sequence set forth in SEQ ID NO. 28; and the CDR3 comprises the sequence set forth in SEQ ID NO. 29; or
based on the Kabat numbering scheme, in the second antigen-binding moiety, the CDR1 comprises the sequence set forth in SEQ ID NO. 30; the CDR2 comprises the sequence set forth in SEQ ID NO. 31; and the CDR3 comprises the sequence set forth in SEQ ID NO. 32; or
based on the Chothia numbering scheme, in the second antigen-binding moiety, the CDR1 comprises the sequence set forth in SEQ ID NO. 39; the CDR2 comprises the sequence set forth in SEQ ID NO. 40; and the CDR3 comprises the sequence set forth in SEQ ID NO. 41; or
based on the Chothia numbering scheme, in the second antigen-binding moiety, the CDR1 comprises the sequence set forth in SEQ ID NO. 42; the CDR2 comprises the sequence set forth in SEQ ID NO. 43; and the CDR3 comprises the sequence set forth in SEQ ID NO. 44; or
based on the Chothia numbering scheme, in the second antigen-binding moiety, the CDR1 comprises the sequence set forth in SEQ ID NO. 45; the CDR2 comprises the sequence set forth in SEQ ID NO. 46; and the CDR3 comprises the sequence set forth in SEQ ID NO. 47; or
based on the Chothia numbering scheme, in the second antigen-binding moiety, the CDR1 comprises the sequence set forth in SEQ ID NO. 48; the CDR2 comprises the sequence set forth in SEQ ID NO. 49; and the CDR3 comprises the sequence set forth in SEQ ID NO. 50; or
based on the IMGT numbering scheme, in the second antigen-binding moiety, the CDR1 comprises the sequence set forth in SEQ ID NO. 57; the CDR2 comprises the sequence set forth in SEQ ID NO. 58; and the CDR3 comprises the sequence set forth in SEQ ID NO. 59; or
based on the IMGT numbering scheme, in the second antigen-binding moiety, the CDR1 comprises the sequence set forth in SEQ ID NO. 60; the CDR2 comprises the sequence set forth in SEQ ID NO. 61; and the CDR3 comprises the sequence set forth in SEQ ID NO. 62; or
based on the IMGT numbering scheme, in the second antigen-binding moiety, the CDR1 comprises the sequence set forth in SEQ ID NO. 63; the CDR2 comprises the sequence set forth in SEQ ID NO. 64; and the CDR3 comprises the sequence set forth in SEQ ID NO. 65; or
based on the IMGT numbering scheme, in the second antigen-binding moiety, the CDR1 comprises the sequence set forth in SEQ ID NO. 66; the CDR2 comprises the sequence set forth in SEQ ID NO. 67; and the CDR3 comprises the sequence set forth in SEQ ID NO. 68.

4. The bispecific antibody according to claim 2, wherein the first antigen-binding moiety comprises an HCDR1, an HCDR2, an HCDR3, an LCDR1, an LCDR2, and an LCDR3 of the following sequences:
(1) based on the Kabat numbering scheme, the sequences set forth in SEQ ID NOs. 15, 16, 17, 18, 19, and 20, respectively; or
(2) based on the Chothia numbering scheme, the sequences set forth in SEQ ID NOs. 33, 34, 35, 36, 37, and 38, respectively; or
(3) based on the IMGT numbering scheme, the sequences set forth in SEQ ID NOs. 51, 52, 53, 54, 55, and 56, respectively; or
(4) sequences having at least 90% identity or having 1, 2, 3, or more amino acid insertions, deletions, and/or substitutions compared with the sequences set forth in (1) to (3) described above, wherein the substitutions are conservative amino acid substitutions.

5. The bispecific antibody according to claim 3, wherein the second antigen-binding moiety comprises a CDR1, a CDR2, and a CDR3 of the following sequences:
(1) based on the Kabat numbering scheme, the sequences set forth in SEQ ID NOs. 21, 22, and 23, respectively; or
(2) based on the Kabat numbering scheme, the sequences set forth in SEQ ID NOs. 24, 25, and 26, respectively; or
(3) based on the Kabat numbering scheme, the sequences set forth in SEQ ID NOs. 27, 28, and 29, respectively; or
(4) based on the Kabat numbering scheme, the sequences set forth in SEQ ID NOs. 30, 31, and 32, respectively; or
(5) based on the Chothia numbering scheme, the sequences set forth in SEQ ID NOs. 39, 40, and 41, respectively; or
(6) based on the Chothia numbering scheme, the sequences set forth in SEQ ID NOs. 42, 43, and 44, respectively; or
(7) based on the Chothia numbering scheme, the sequences set forth in SEQ ID NOs. 45, 46, and 47, respectively; or
(8) based on the Chothia numbering scheme, the sequences set forth in SEQ ID NOs. 48, 49, and 50, respectively; or
(9) based on the IMGT numbering scheme, the sequences set forth in SEQ ID NOs. 57, 58, and 59, respectively; or
(10) based on the IMGT numbering scheme, the sequences set forth in SEQ ID NOs. 60, 61, and 62, respectively; or
(11) based on the IMGT numbering scheme, the sequences set forth in SEQ ID NOs. 63, 64, and 65, respectively; or
(12) based on the IMGT numbering scheme, the sequences set forth in SEQ ID NOs. 66, 67, and 68, respectively; or
(13) sequences having at least 90% identity or having 1, 2, 3, or more amino acid insertions, deletions, and/or substitutions compared with the sequences set forth in (1) to (12) described above, wherein the substitutions are conservative amino acid substitutions.

6. The bispecific antibody according to claim 1, wherein the VH of the first antigen-binding moiety comprises a sequence having at least 90% identity to the amino acid sequence set forth in SEQ ID NO. 9; the VL of the first antigen-binding moiety comprises a sequence having at least 90% identity to the amino acid sequence set forth in SEQ ID NO. 10.

7. The bispecific antibody according to claim 1, wherein the second antigen-binding moiety comprises a sequence having at least 90% identity to the amino acid sequence set forth in any one of SEQ ID NOs. 11-14.

8. The bispecific antibody according to claim 1, wherein the bispecific antibody comprises: a heavy chain comprising the anti-CD3 VH, a light chain comprising the anti-CD3 VL, and a heavy chain comprising the anti-MUC17 VHH.

9. The bispecific antibody according to claim 8, wherein the heavy chain comprising the anti-CD3 VH comprises a sequence having at least 80% identity to the amino acid sequence set forth in SEQ ID NO. 1, the light chain comprising the anti-CD3 VL comprises a sequence having at least 80% identity to the amino acid sequence set forth in SEQ ID NO. 2, and the heavy chain comprising the anti-MUC17 VHH comprises a sequence having at least 80% identity to the amino acid sequence set forth in SEQ ID NO. 3, 4, 5, or 6.

10. The bispecific antibody according to claim 1, wherein the bispecific antibody is a humanized antibody.

11. The bispecific antibody according to claim 1, wherein the bispecific antibody binds to human and monkey MUC17 with a KD superior to 1.00E-9 M.

12. An isolated nucleic acid molecule encoding the bispecific antibody according to claim 1.

13. A vector, comprising the nucleic acid molecule according to claim 12.

14. A host cell, comprising the vector according to claim 13, wherein the cell is a prokaryotic cell.

15. (canceled)

16. A pharmaceutical composition, comprising the bispecific antibody according to claim 1, or a nucleic acid molecule encoding the bispecific antibody, or a vector comprising the nucleic acid molecule, or the host cell comprising the vector, and a pharmaceutically acceptable carrier.

17. The pharmaceutical composition according to claim 16, further comprising an additional therapeutic agent, wherein the additional therapeutic agent is an antineoplastic agent.

18-21. (canceled)

22. A method for treating a cancer, a tumor, or an infectious disease, comprising administering to a patient in need thereof an effective amount of the bispecific antibody according to claim 1, or a nucleic acid molecule encoding the bispecific antibody, or a vector comprising the nucleic acid molecule, or a host cell comprising the vector, or the pharmaceutical composition comprising the bispecific antibody, wherein the cancer or tumor is selected from a solid tumor and a hematological tumor.

23. The method according to claim 22, wherein the cancer or tumor is a MUC17-positive cancer or MUC17-positive tumor.

24. The method according to claim 22, wherein the cancer or tumor is selected from gastric cancer, pancreatic cancer, small intestine cancer, large intestine cancer, rectal cancer, colon cancer, colorectal cancer, esophageal cancer, breast cancer, non-small cell lung cancer, adenocarcinoma, non-Hodgkin's lymphoma (NHL), B-cell lymphoma, B-cell leukemia, multiple myeloma, renal cancer, prostate cancer, liver cancer, head and neck cancer, melanoma, ovarian cancer, mesothelioma, glioblastoma, thyroid cancer, bladder cancer, cervical cancer, blood cancer, skin cancer, epithelial cancer, brain cancer, and central nervous system cancer.

25-27. (canceled)

28. The pharmaceutical composition according to claim 17, wherein the antineoplastic agent is a PD-1 axis binding antagonist, a small molecule antineoplastic agent, or a cell therapeutic agent.
